SEQUENCE LISTING (230 sequences)

SEQ ID NO: 1 - DNA, 19 bp, synthetic construct
  source 1..19: mol_type = other DNA
ttcagctggttggggcctc

SEQ ID NO: 2 - DNA, 19 bp, synthetic construct
  source 1..19: mol_type = other DNA
ggttgctctgctttcttca

SEQ ID NO: 3 - DNA, 19 bp, synthetic construct
  source 1..19: mol_type = other DNA
ggctgccagctcctgggtt

SEQ ID NO: 4 - DNA, 19 bp, synthetic construct
  source 1..19: mol_type = other DNA
gacgcctgcactgtttttg

SEQ ID NO: 5 - DNA, 19 bp, synthetic construct
  source 1..19: mol_type = other DNA
agcgctggggtcacagacg

SEQ ID NO: 6 - DNA, 19 bp, synthetic construct
  source 1..19: mol_type = other DNA
cgacgctgccacgacagcg

SEQ ID NO: 7 - DNA, 19 bp, synthetic construct
  source 1..19: mol_type = other DNA
ctttcttcagctggttggg

SEQ ID NO: 8 - DNA, 19 bp, synthetic construct
  source 1..19: mol_type = other DNA
tcctgggttgctctgcttt

SEQ ID NO: 9 - DNA, 19 bp, synthetic construct
  source 1..19: mol_type = other DNA
accgaggctgccagctcct

SEQ ID NO: 10 - DNA, 19 bp, synthetic construct
  source 1..19: mol_type = other DNA
tcacagacgcctgcactgt

SEQ ID NO: 11 - DNA, 19 bp, synthetic construct
  source 1..19: mol_type = other DNA
acgacagcgctggggtcac

SEQ ID NO: 12 - DNA, 19 bp, synthetic construct
  source 1..19: mol_type = other DNA
ctctgctttcttcagctgg

SEQ ID NO: 13 - DNA, 19 bp, synthetic construct
  source 1..19: mol_type = other DNA
ccagctcctgggttgctct

SEQ ID NO: 14 - DNA, 19 bp, synthetic construct
  source 1..19: mol_type = other DNA
ctgcactgtttttgcagac

SEQ ID NO: 15 - DNA, 19 bp, synthetic construct
  source 1..19: mol_type = other DNA
tggggtcacagacgcctgc

SEQ ID NO: 16 - DNA, 19 bp, synthetic construct
  source 1..19: mol_type = other DNA
ctgccacgacagcgctggg

SEQ ID NO: 17 - DNA, 19 bp, synthetic construct
  source 1..19: mol_type = other DNA
tctgcggtccgttggcggg

SEQ ID NO: 18 - DNA, 19 bp, synthetic construct
  source 1..19: mol_type = other DNA
cggggtcattgatctgggc

SEQ ID NO: 19 - DNA, 19 bp, synthetic construct
  source 1..19: mol_type = other DNA
gggcgggcccttagacact

SEQ ID NO: 20 - DNA, 19 bp, synthetic construct
  source 1..19: mol_type = other DNA
gcctcagccactgtcccat

SEQ ID NO: 21 - DNA, 19 bp, synthetic construct
  source 1..19: mol_type = other DNA
ccatgctggagctggctgt

SEQ ID NO: 22 - DNA, 19 bp, synthetic construct
  source 1..19: mol_type = other DNA
ctgtgtttctgctgtcgtc

SEQ ID NO: 23 - DNA, 19 bp, synthetic construct
  source 1..19: mol_type = other DNA
agcactggatagaggagac

SEQ ID NO: 24 - DNA, 19 bp, synthetic construct
  source 1..19: mol_type = other DNA
agacacctggcggaagcct

SEQ ID NO: 25 - DNA, 19 bp, synthetic construct
  source 1..19: mol_type = other DNA
ggtccgttggcggggtcat

SEQ ID NO: 26 - DNA, 19 bp, synthetic construct
  source 1..19: mol_type = other DNA
tcattgatctgggcgggcc

SEQ ID NO: 27 - DNA, 19 bp, synthetic construct
  source 1..19: mol_type = other DNA
ggcccttagacacttccgg

SEQ ID NO: 28 - DNA, 19 bp, synthetic construct
  source 1..19: mol_type = other DNA
agccactgtcccatgctgg

SEQ ID NO: 29 - DNA, 19 bp, synthetic construct
  source 1..19: mol_type = other DNA
ctggagctggctgtgtttc

SEQ ID NO: 30 - DNA, 19 bp, synthetic construct
  source 1..19: mol_type = other DNA
ccggctgggcagcactgga

SEQ ID NO: 31 - DNA, 19 bp, synthetic construct
  source 1..19: mol_type = other DNA
tggatagaggagacacctg

SEQ ID NO: 32 - DNA, 19 bp, synthetic construct
  source 1..19: mol_type = other DNA
cctggcggaagccttggtt

SEQ ID NO: 33 - DNA, 19 bp, synthetic construct
  source 1..19: mol_type = other DNA
gttggcggggtcattgatc

SEQ ID NO: 34 - DNA, 19 bp, synthetic construct
  source 1..19: mol_type = other DNA
gatctgggcgggcccttag

SEQ ID NO: 35 - DNA, 19 bp, synthetic construct
  source 1..19: mol_type = other DNA
tgtgagcctcagccactgt

SEQ ID NO: 36 - DNA, 19 bp, synthetic construct
  source 1..19: mol_type = other DNA
ctgtcccatgctggagctg

SEQ ID NO: 37 - DNA, 19 bp, synthetic construct
  source 1..19: mol_type = other DNA
gctggctgtgtttctgctg

SEQ ID NO: 38 - DNA, 19 bp, synthetic construct
  source 1..19: mol_type = other DNA
tgggcagcactggatagag

SEQ ID NO: 39 - DNA, 19 bp, synthetic construct
  source 1..19: mol_type = other DNA
agaggagacacctggcgga

SEQ ID NO: 40 - DNA, 19 bp, synthetic construct
  source 1..19: mol_type = other DNA
cggaagccttggtttcctt

SEQ ID NO: 41 - DNA, 19 bp, synthetic construct
  source 1..19: mol_type = other DNA
cttcttggtcctgggctgg

SEQ ID NO: 42 - DNA, 19 bp, synthetic construct
  source 1..19: mol_type = other DNA
ctgggagtggaggctccat

SEQ ID NO: 43 - DNA, 19 bp, synthetic construct
  source 1..19: mol_type = other DNA
ccatcctggcctggatggg

SEQ ID NO: 44 - DNA, 19 bp, synthetic construct
  source 1..19: mol_type = other DNA
tggtcctgggctgggagtg

SEQ ID NO: 45 - DNA, 19 bp, synthetic construct
  source 1..19: mol_type = other DNA
agtggaggctccatcctgg

SEQ ID NO: 46 - DNA, 19 bp, synthetic construct
  source 1..19: mol_type = other DNA
ctggcctggatggggtggc

SEQ ID NO: 47 - DNA, 19 bp, synthetic construct
  source 1..19: mol_type = other DNA
ctgggctgggagtggaggc

SEQ ID NO: 48 - DNA, 19 bp, synthetic construct
  source 1..19: mol_type = other DNA
aggctccatcctggcctgg

SEQ ID NO: 49 - DNA, 19 bp, synthetic construct
  source 1..19: mol_type = other DNA
gagacggagtctcgctctg

SEQ ID NO: 50 - DNA, 19 bp, synthetic construct
  source 1..19: mol_type = other DNA
tctgtcgcccaggctggag

SEQ ID NO: 51 - DNA, 19 bp, synthetic construct
  source 1..19: mol_type = other DNA
ggagtgcagtggtgcgatc

SEQ ID NO: 52 - DNA, 19 bp, synthetic construct
  source 1..19: mol_type = other DNA
ggagtctcgctctgtcgcc

SEQ ID NO: 53 - DNA, 19 bp, synthetic construct
  source 1..19: mol_type = other DNA
cgcccaggctggagtgcag

SEQ ID NO: 54 - DNA, 19 bp, synthetic construct
  source 1..19: mol_type = other DNA
gcagtggtgcgatctcagc

SEQ ID NO: 55 - DNA, 19 bp, synthetic construct
  source 1..19: mol_type = other DNA
ctcgctctgtcgcccaggc

SEQ ID NO: 56 - DNA, 19 bp, synthetic construct
  source 1..19: mol_type = other DNA
aggctggagtgcagtggtg

SEQ ID NO: 57 - DNA, 19 bp, synthetic construct
  source 1..19: mol_type = other DNA
tggaaggctgaggctgcag

SEQ ID NO: 58 - DNA, 19 bp, synthetic construct
  source 1..19: mol_type = other DNA
gcagtgagccgagattgca

SEQ ID NO: 59 - DNA, 19 bp, synthetic construct
  source 1..19: mol_type = other DNA
tgcaccactgcactccagc

SEQ ID NO: 60 - DNA, 19 bp, synthetic construct
  source 1..19: mol_type = other DNA
ggctgaggctgcagtgagc

SEQ ID NO: 61 - DNA, 19 bp, synthetic construct
  source 1..19: mol_type = other DNA
gagccgagattgcaccact

SEQ ID NO: 62 - DNA, 19 bp, synthetic construct
  source 1..19: mol_type = other DNA
cactgcactccagcctggg

SEQ ID NO: 63 - DNA, 19 bp, synthetic construct
  source 1..19: mol_type = other DNA
aggctgcagtgagccgaga

SEQ ID NO: 64 - DNA, 19 bp, synthetic construct
  source 1..19: mol_type = other DNA
gagattgcaccactgcact

SEQ ID NO: 65 - DNA, 19 bp, synthetic construct
  source 1..19: mol_type = other DNA
gggcgtgggagccaggcct

SEQ ID NO: 66 - DNA, 19 bp, synthetic construct
  source 1..19: mol_type = other DNA
gcctcagtcctggacgagg

SEQ ID NO: 67 - DNA, 19 bp, synthetic construct
  source 1..19: mol_type = other DNA
gagggtggttggaaactga

SEQ ID NO: 68 - DNA, 19 bp, synthetic construct
  source 1..19: mol_type = other DNA
tgggagccaggcctcagtc

SEQ ID NO: 69 - DNA, 19 bp, synthetic construct
  source 1..19: mol_type = other DNA
agtcctggacgagggtggt

SEQ ID NO: 70 - DNA, 19 bp, synthetic construct
  source 1..19: mol_type = other DNA
tggttggaaactgatggag

SEQ ID NO: 71 - DNA, 19 bp, synthetic construct
  source 1..19: mol_type = other DNA
gccaggcctcagtcctgga

SEQ ID NO: 72 - DNA, 19 bp, synthetic construct
  source 1..19: mol_type = other DNA
tggacgagggtggttggaa

SEQ ID NO: 73 - DNA, 19 bp, synthetic construct
  source 1..19: mol_type = other DNA
aatggcagattcaggtata

SEQ ID NO: 74 - DNA, 19 bp, synthetic construct
  source 1..19: mol_type = other DNA
aatgaaatggcagattcag

SEQ ID NO: 75 - DNA, 19 bp, synthetic construct
  source 1..19: mol_type = other DNA
aggggatgacacagctcct

SEQ ID NO: 76 - DNA, 19 bp, synthetic construct
  source 1..19: mol_type = other DNA
cgggtggcgcagagcaggg

SEQ ID NO: 77 - DNA, 19 bp, synthetic construct
  source 1..19: mol_type = other DNA
cgggacctggcacagcggg

SEQ ID NO: 78 - DNA, 19 bp, synthetic construct
  source 1..19: mol_type = other DNA
gcaactgctgtgtgtcggg

SEQ ID NO: 79 - DNA, 19 bp, synthetic construct
  source 1..19: mol_type = other DNA
agcggcgtatggggagcaa

SEQ ID NO: 80 - DNA, 19 bp, synthetic construct
  source 1..19: mol_type = other DNA
accggccgaccgcagagcg

SEQ ID NO: 81 - DNA, 19 bp, synthetic construct
  source 1..19: mol_type = other DNA
agagcaggggatgacacag

SEQ ID NO: 82 - DNA, 19 bp, synthetic construct
  source 1..19: mol_type = other DNA
cacagcgggtggcgcagag

SEQ ID NO: 83 - DNA, 19 bp, synthetic construct
  source 1..19: mol_type = other DNA
tgtgtcgggacctggcaca

SEQ ID NO: 84 - DNA, 19 bp, synthetic construct
  source 1..19: mol_type = other DNA
ggggagcaactgctgtgtg

SEQ ID NO: 85 - DNA, 19 bp, synthetic construct
  source 1..19: mol_type = other DNA
cgcagagcggcgtatgggg

SEQ ID NO: 86 - DNA, 19 bp, synthetic construct
  source 1..19: mol_type = other DNA
gatgcaccggccgaccgca

SEQ ID NO: 87 - DNA, 19 bp, synthetic construct
  source 1..19: mol_type = other DNA
ggcgcagagcaggggatga

SEQ ID NO: 88 - DNA, 19 bp, synthetic construct
  source 1..19: mol_type = other DNA
cctggcacagcgggtggcg

SEQ ID NO: 89 - DNA, 19 bp, synthetic construct
  source 1..19: mol_type = other DNA
tgctgtgtgtcgggacctg

SEQ ID NO: 90 - DNA, 19 bp, synthetic construct
  source 1..19: mol_type = other DNA
cgtatggggagcaactgct

SEQ ID NO: 91 - DNA, 19 bp, synthetic construct
  source 1..19: mol_type = other DNA
ccgaccgcagagcggcgta

SEQ ID NO: 92 - DNA, 19 bp, synthetic construct
  source 1..19: mol_type = other DNA
ggccggatgcaccggccga

SEQ ID NO: 93 - DNA, 19 bp, synthetic construct
  source 1..19: mol_type = other DNA
acggaacctctgggcacaa

SEQ ID NO: 94 - DNA, 19 bp, synthetic construct
  source 1..19: mol_type = other DNA
gcctgtcttctaggcacgg

SEQ ID NO: 95 - DNA, 19 bp, synthetic construct
  source 1..19: mol_type = other DNA
gcttcatgccagcctgcct

SEQ ID NO: 96 - DNA, 19 bp, synthetic construct
  source 1..19: mol_type = other DNA
taggcacggaacctctggg

SEQ ID NO: 97 - DNA, 19 bp, synthetic construct
  source 1..19: mol_type = other DNA
agcctgcctgtcttctagg

SEQ ID NO: 98 - DNA, 19 bp, synthetic construct
  source 1..19: mol_type = other DNA
catgtgcttcatgccagcc

SEQ ID NO: 99 - DNA, 19 bp, synthetic construct
  source 1..19: mol_type = other DNA
tcttctaggcacggaacct

SEQ ID NO: 100 - DNA, 19 bp, synthetic construct
  source 1..19: mol_type = other DNA
atgccagcctgcctgtctt

SEQ ID NO: 101 - DNA, 19 bp, synthetic construct
  source 1..19: mol_type = other DNA
cccacatccttgtcctgca

SEQ ID NO: 102 - DNA, 19 bp, synthetic construct
  source 1..19: mol_type = other DNA
gtgaggaggggccccgtgg

SEQ ID NO: 103 - DNA, 19 bp, synthetic construct
  source 1..19: mol_type = other DNA
atccttgtcctgcacagcc

SEQ ID NO: 104 - DNA, 19 bp, synthetic construct
  source 1..19: mol_type = other DNA
tccaagtgaggaggggccc

SEQ ID NO: 105 - DNA, 19 bp, synthetic construct
  source 1..19: mol_type = other DNA
tggcagccgagcccgaggc

SEQ ID NO: 106 - DNA, 19 bp, synthetic construct
  source 1..19: mol_type = other DNA
aggcacaggggagaggcca

SEQ ID NO: 107 - DNA, 19 bp, synthetic construct
  source 1..19: mol_type = other DNA
gccagggtggcaaggcttg

SEQ ID NO: 108 - DNA, 19 bp, synthetic construct
  source 1..19: mol_type = other DNA
ttggggtccttgccagggg

SEQ ID NO: 109 - DNA, 19 bp, synthetic construct
  source 1..19: mol_type = other DNA
ggggagtggtctgaggcca

SEQ ID NO: 110 - DNA, 19 bp, synthetic construct
  source 1..19: mol_type = other DNA
gccatctccaccaggagcc

SEQ ID NO: 111 - DNA, 19 bp, synthetic construct
  source 1..19: mol_type = other DNA
gccgagcccgaggcacagg

SEQ ID NO: 112 - DNA, 19 bp, synthetic construct
  source 1..19: mol_type = other DNA
caggggagaggccagggtg

SEQ ID NO: 113 - DNA, 19 bp, synthetic construct
  source 1..19: mol_type = other DNA
ggtggcaaggcttgtggca

SEQ ID NO: 114 - DNA, 19 bp, synthetic construct
  source 1..19: mol_type = other DNA
gtccttgccaggggagtgg

SEQ ID NO: 115 - DNA, 19 bp, synthetic construct
  source 1..19: mol_type = other DNA
gtggtctgaggccatctcc

SEQ ID NO: 116 - DNA, 19 bp, synthetic construct
  source 1..19: mol_type = other DNA
gcccgaggcacaggggaga

SEQ ID NO: 117 - DNA, 19 bp, synthetic construct
  source 1..19: mol_type = other DNA
gagaggccagggtggcaag

SEQ ID NO: 118 - DNA, 19 bp, synthetic construct
  source 1..19: mol_type = other DNA
ccctcttggggtccttgcc

SEQ ID NO: 119 - DNA, 19 bp, synthetic construct
  source 1..19: mol_type = other DNA
tgccaggggagtggtctga

SEQ ID NO: 120 - DNA, 19 bp, synthetic construct
  source 1..19: mol_type = other DNA
ctgaggccatctccaccag

SEQ ID NO: 121 - DNA, 19 bp, synthetic construct
  source 1..19: mol_type = other DNA
cgagcggcccgtgggagat

SEQ ID NO: 122 - DNA, 19 bp, synthetic construct
  source 1..19: mol_type = other DNA
agatggagagaggagagca

SEQ ID NO: 123 - DNA, 19 bp, synthetic construct
  source 1..19: mol_type = other DNA
agcaggacgggaggagctg

SEQ ID NO: 124 - DNA, 19 bp, synthetic construct
  source 1..19: mol_type = other DNA
ggcccgtgggagatggaga

SEQ ID NO: 125 - DNA, 19 bp, synthetic construct
  source 1..19: mol_type = other DNA
gagagaggagagcaggacg

SEQ ID NO: 126 - DNA, 19 bp, synthetic construct
  source 1..19: mol_type = other DNA
gacgggaggagctggcagt

SEQ ID NO: 127 - DNA, 19 bp, synthetic construct
  source 1..19: mol_type = other DNA
gtgggagatggagagagga

SEQ ID NO: 128 - DNA, 19 bp, synthetic construct
  source 1..19: mol_type = other DNA
aggagagcaggacgggagg

SEQ ID NO: 129 - DNA, 19 bp, synthetic construct
  source 1..19: mol_type = other DNA
ttcgaggccagcctcgcca

SEQ ID NO: 130 - DNA, 19 bp, synthetic construct
  source 1..19: mol_type = other DNA
acttgaggctgggagttcg

SEQ ID NO: 131 - DNA, 19 bp, synthetic construct
  source 1..19: mol_type = other DNA
cgaggcaggcagatcactt

SEQ ID NO: 132 - DNA, 19 bp, synthetic construct
  source 1..19: mol_type = other DNA
gggagttcgaggccagcct

SEQ ID NO: 133 - DNA, 19 bp, synthetic construct
  source 1..19: mol_type = other DNA
agatcacttgaggctggga

SEQ ID NO: 134 - DNA, 19 bp, synthetic construct
  source 1..19: mol_type = other DNA
ggaggcgaggcaggcagat

SEQ ID NO: 135 - DNA, 19 bp, synthetic construct
  source 1..19: mol_type = other DNA
aggctgggagttcgaggcc

SEQ ID NO: 136 - DNA, 19 bp, synthetic construct
  source 1..19: mol_type = other DNA
caggcagatcacttgaggc

SEQ ID NO: 137 - DNA, 19 bp, synthetic construct
  source 1..19: mol_type = other DNA
gctgtggggaggaggctcc

SEQ ID NO: 138 - DNA, 19 bp, synthetic construct
  source 1..19: mol_type = other DNA
ctgtctaggactcaggctg

SEQ ID NO: 139 - DNA, 19 bp, synthetic construct
  source 1..19: mol_type = other DNA
ggatgcagaggtgggctgt

SEQ ID NO: 140 - DNA, 19 bp, synthetic construct
  source 1..19: mol_type = other DNA
ctcaggctgtggggaggag

SEQ ID NO: 141 - DNA, 19 bp, synthetic construct
  source 1..19: mol_type = other DNA
gtgggctgtctaggactca

SEQ ID NO: 142 - DNA, 19 bp, synthetic construct
  source 1..19: mol_type = other DNA
gggcaggatgcagaggtgg

SEQ ID NO: 143 - DNA, 19 bp, synthetic construct
  source 1..19: mol_type = other DNA
taggactcaggctgtgggg

SEQ ID NO: 144 - DNA, 19 bp, synthetic construct
  source 1..19: mol_type = other DNA
cagaggtgggctgtctagg

SEQ ID NO: 145 - DNA, 19 bp, synthetic construct
  source 1..19: mol_type = other DNA
gcagcagtgaggacagcag

SEQ ID NO: 146 - DNA, 19 bp, synthetic construct
  source 1..19: mol_type = other DNA
agtgaggacagcagggagg

SEQ ID NO: 147 - DNA, 19 bp, synthetic construct
  source 1..19: mol_type = other DNA
ggggctggagggccatgag

SEQ ID NO: 148 - DNA, 19 bp, synthetic construct
  source 1..19: mol_type = other DNA
tggagggccatgagcagag

SEQ ID NO: 149 - DNA, 19 bp, synthetic construct
  source 1..19: mol_type = other DNA
agagccggctgaggctggg

SEQ ID NO: 150 - DNA, 19 bp, synthetic construct
  source 1..19: mol_type = other DNA
tgggcctccagtgtcaggg

SEQ ID NO: 151 - DNA, 19 bp, synthetic construct
  source 1..19: mol_type = other DNA
aggggttcctgggggtgtg

SEQ ID NO: 152 - DNA, 19 bp, synthetic construct
  source 1..19: mol_type = other DNA
cggctgaggctgggcctcc

SEQ ID NO: 153 - DNA, 19 bp, synthetic construct
  source 1..19: mol_type = other DNA
ctccagtgtcaggggttcc

SEQ ID NO: 154 - DNA, 19 bp, synthetic construct
  source 1..19: mol_type = other DNA
ttcctgggggtgtggcagg

SEQ ID NO: 155 - DNA, 19 bp, synthetic construct
  source 1..19: mol_type = other DNA
gaggctgggcctccagtgt

SEQ ID NO: 156 - DNA, 19 bp, synthetic construct
  source 1..19: mol_type = other DNA
gtgtcaggggttcctgggg

SEQ ID NO: 157 - DNA, 19 bp, synthetic construct
  source 1..19: mol_type = other DNA
cctcagcactttcagaggc

SEQ ID NO: 158 - DNA, 19 bp, synthetic construct
  source 1..19: mol_type = other DNA
aggctgaggcaggaggatc

SEQ ID NO: 159 - DNA, 19 bp, synthetic construct
  source 1..19: mol_type = other DNA
gatcaattgaggccaggag

SEQ ID NO: 160 - DNA, 19 bp, synthetic construct
  source 1..19: mol_type = other DNA
gcactttcagaggctgagg

SEQ ID NO: 161 - DNA, 19 bp, synthetic construct
  source 1..19: mol_type = other DNA
gaggcaggaggatcaattg

SEQ ID NO: 162 - DNA, 19 bp, synthetic construct
  source 1..19: mol_type = other DNA
attgaggccaggagtttaa

SEQ ID NO: 163 - DNA, 19 bp, synthetic construct
  source 1..19: mol_type = other DNA
ttcagaggctgaggcagga

SEQ ID NO: 164 - DNA, 19 bp, synthetic construct
  source 1..19: mol_type = other DNA
aggaggatcaattgaggcc

SEQ ID NO: 165 - DNA, 19 bp, synthetic construct
  source 1..19: mol_type = other DNA
aagcttcttggttgagaac

SEQ ID NO: 166 - DNA, 19 bp, synthetic construct
  source 1..19: mol_type = other DNA
agaaggttgtgagacaagc

SEQ ID NO: 167 - DNA, 19 bp, synthetic construct
  source 1..19: mol_type = other DNA
tggggaagcagatgcagaa

SEQ ID NO: 168 - DNA, 19 bp, synthetic construct
  source 1..19: mol_type = other DNA
gagacaagcttcttggttg

SEQ ID NO: 169 - DNA, 19 bp, synthetic construct
  source 1..19: mol_type = other DNA
gatgcagaaggttgtgaga

SEQ ID NO: 170 - DNA, 19 bp, synthetic construct
  source 1..19: mol_type = other DNA
tattctggggaagcagatg

SEQ ID NO: 171 - DNA, 19 bp, synthetic construct
  source 1..19: mol_type = other DNA
gttgtgagacaagcttctt

SEQ ID NO: 172 - DNA, 19 bp, synthetic construct
  source 1..19: mol_type = other DNA
aagcagatgcagaaggttg

SEQ ID NO: 173 - DNA, 19 bp, synthetic construct
  source 1..19: mol_type = other DNA
caaaacatgaaattcaata

SEQ ID NO: 174 - DNA, 19 bp, synthetic construct
  source 1..19: mol_type = other DNA
aaatccaaaacatgaaatt

SEQ ID NO: 175 - DNA, 19 bp, synthetic construct
  source 1..19: mol_type = other DNA
tcaggtcaccacggaaggg

SEQ ID NO: 176 - DNA, 19 bp, synthetic construct
  source 1..19: mol_type = other DNA
gagtctgtagggctctcag

SEQ ID NO: 177 - DNA, 19 bp, synthetic construct
  source 1..19: mol_type = other DNA
agtctctgggccacagagt

SEQ ID NO: 178 - DNA, 19 bp, synthetic construct
  source 1..19: mol_type = other DNA
ggtcggaggtcacggagtc

SEQ ID NO: 179 - DNA, 19 bp, synthetic construct
  source 1..19: mol_type = other DNA
ctgggtctgggatggggtc

SEQ ID NO: 180 - DNA, 19 bp, synthetic construct
  source 1..19: mol_type = other DNA
tgacatggggggggcctgg

SEQ ID NO: 181 - DNA, 19 bp, synthetic construct
  source 1..19: mol_type = other DNA
ggctctcaggtcaccacgg

SEQ ID NO: 182 - DNA, 19 bp, synthetic construct
  source 1..19: mol_type = other DNA
ccacagagtctgtagggct

SEQ ID NO: 183 - DNA, 19 bp, synthetic construct
  source 1..19: mol_type = other DNA
cacggagtctctgggccac

SEQ ID NO: 184 - DNA, 19 bp, synthetic construct
  source 1..19: mol_type = other DNA
gatggggtcggaggtcacg

SEQ ID NO: 185 - DNA, 19 bp, synthetic construct
  source 1..19: mol_type = other DNA
ggggcctgggtctgggatg

SEQ ID NO: 186 - DNA, 19 bp, synthetic construct
  source 1..19: mol_type = other DNA
ggctgtgacatgggggggg

SEQ ID NO: 187 - DNA, 19 bp, synthetic construct
  source 1..19: mol_type = other DNA
tgtagggctctcaggtcac

SEQ ID NO: 188 - DNA, 19 bp, synthetic construct
  source 1..19: mol_type = other DNA
ctgggccacagagtctgta

SEQ ID NO: 189 - DNA, 19 bp, synthetic construct
  source 1..19: mol_type = other DNA
gaggtcacggagtctctgg

SEQ ID NO: 190 - DNA, 19 bp, synthetic construct
  source 1..19: mol_type = other DNA
tctgggatggggtcggagg

SEQ ID NO: 191 - DNA, 19 bp, synthetic construct
  source 1..19: mol_type = other DNA
tggggggggcctgggtctg

SEQ ID NO: 192 - DNA, 19 bp, synthetic construct
  source 1..19: mol_type = other DNA
ctgcgggctgtgacatggg

SEQ ID NO: 193 - DNA, 19 bp, synthetic construct
  source 1..19: mol_type = other DNA
aggtggtgggccggtgatc

SEQ ID NO: 194 - DNA, 19 bp, synthetic construct
  source 1..19: mol_type = other DNA
gaggcaggtggtgggccgg

SEQ ID NO: 195 - DNA, 19 bp, synthetic construct
  source 1..19: mol_type = other DNA
cacagcagtggcaaggagc

SEQ ID NO: 196 - DNA, 19 bp, synthetic construct
  source 1..19: mol_type = other DNA
gagcgctggggagccacca

SEQ ID NO: 197 - DNA, 19 bp, synthetic construct
  source 1..19: mol_type = other DNA
accagctgtgcctcatcct

SEQ ID NO: 198 - DNA, 19 bp, synthetic construct
  source 1..19: mol_type = other DNA
cagtggcaaggagcgctgg

SEQ ID NO: 199 - DNA, 19 bp, synthetic construct
  source 1..19: mol_type = other DNA
ctggggagccaccagctgt

SEQ ID NO: 200 - DNA, 19 bp, synthetic construct
  source 1..19: mol_type = other DNA
ctgtgcctcatcctctctg

SEQ ID NO: 201 - DNA, 19 bp, synthetic construct
  source 1..19: mol_type = other DNA
gcaaggagcgctggggagc

SEQ ID NO: 202 - DNA, 19 bp, synthetic construct
  source 1..19: mol_type = other DNA
gagccaccagctgtgcctc

SEQ ID NO: 203 - DNA, 19 bp, synthetic construct
  source 1..19: mol_type = other DNA
agcagaaggttttgctcct

SEQ ID NO: 204 - DNA, 19 bp, synthetic construct
  source 1..19: mol_type = other DNA
tcagtagcagaaggttttg

SEQ ID NO: 205 - DNA, 19 bp, synthetic construct
  source 1..19: mol_type = other DNA
actcaggaggctgaggtgg

SEQ ID NO: 206 - DNA, 19 bp, synthetic construct
  source 1..19: mol_type = other DNA
gtgggaggatcacttgggc

SEQ ID NO: 207 - DNA, 19 bp, synthetic construct
  source 1..19: mol_type = other DNA
gggcctgggacgtcaaggc

SEQ ID NO: 208 - DNA, 19 bp, synthetic construct
  source 1..19: mol_type = other DNA
ggaggctgaggtgggagga

SEQ ID NO: 209 - DNA, 19 bp, synthetic construct
  source 1..19: mol_type = other DNA
aggatcacttgggcctggg

SEQ ID NO: 210 - DNA, 19 bp, synthetic construct
  source 1..19: mol_type = other DNA
tgggacgtcaaggctacag

SEQ ID NO: 211 - DNA, 19 bp, synthetic construct
  source 1..19: mol_type = other DNA
ctgaggtgggaggatcact

SEQ ID NO: 212 - DNA, 19 bp, synthetic construct
  source 1..19: mol_type = other DNA
cacttgggcctgggacgtc

SEQ ID NO: 213 - DNA, 19 bp, synthetic construct
  source 1..19: mol_type = other DNA
atgtctttaaagagcctgt

SEQ ID NO: 214 - DNA, 19 bp, synthetic construct
  source 1..19: mol_type = other DNA
ctgtcctgatctggcctct

SEQ ID NO: 215 - DNA, 19 bp, synthetic construct
  source 1..19: mol_type = other DNA
ctctccaggcagggtgagc

SEQ ID NO: 216 - DNA, 19 bp, synthetic construct
  source 1..19: mol_type = other DNA
tttaaagagcctgtcctga

SEQ ID NO: 217 - DNA, 19 bp, synthetic construct
  source 1..19: mol_type = other DNA
ctgatctggcctctccagg

SEQ ID NO: 218 - DNA, 19 bp, synthetic construct
  source 1..19: mol_type = other DNA
caggcagggtgagctcttt

SEQ ID NO: 219 - DNA, 19 bp, synthetic construct
  source 1..19: mol_type = other DNA
agagcctgtcctgatctgg

SEQ ID NO: 220 - DNA, 19 bp, synthetic construct
  source 1..19: mol_type = other DNA
ctggcctctccaggcaggg

SEQ ID NO: 221 - DNA, 19 bp, synthetic construct
  source 1..19: mol_type = other DNA
ctctcctctccacggctcc

SEQ ID NO: 222 - DNA, 19 bp, synthetic construct
  source 1..19: mol_type = other DNA
tttctctctcctctccacg

SEQ ID NO: 223 - DNA, 19 bp, synthetic construct
  source 1..19: mol_type = other DNA
tgtgaggatgaacggaggt

SEQ ID NO: 224 - DNA, 19 bp, synthetic construct
  source 1..19: mol_type = other DNA
tccatcaggtgggggtgtg

SEQ ID NO: 225 - DNA, 19 bp, synthetic construct
  source 1..19: mol_type = other DNA
agattataatagatatcca

SEQ ID NO: 226 - DNA, 19 bp, synthetic construct
  source 1..19: mol_type = other DNA
gggggtgtgaggatgaacg

SEQ ID NO: 227 - DNA, 19 bp, synthetic construct
  source 1..19: mol_type = other DNA
agatatccatcaggtgggg

SEQ ID NO: 228 - DNA, 19 bp, synthetic construct
  source 1..19: mol_type = other DNA
ggtggagattataatagat

SEQ ID NO: 229 - DNA, 19 bp, synthetic construct
  source 1..19: mol_type = other DNA
caggtgggggtgtgaggat

SEQ ID NO: 230 - DNA, 19 bp, synthetic construct
  source 1..19: mol_type = other DNA
ataatagatatccatcagg

(19) United States
(12) Patent Application Publication
(10) Pub. No.: US 2025/0034569 A1
(43) Pub. Date: Jan. 30, 2025

(54) ANTISENSE OLIGONUCLEOTIDE DRUG TARGETS

(71) Applicant: PRESIDENT AND FELLOWS OF HARVARD COLLEGE, Cambridge, MA (US)

(72) Inventors: Craig L. BOHRSON, Cambridge, MA (US); Peter J. PARK, Cambridge, MA (US); Jin Kuk KIM, Cambridge, MA (US)

(73) Assignee: PRESIDENT AND FELLOWS OF HARVARD COLLEGE, Cambridge, MA (US)

(21) Appl. No.: 18/716,754

(22) PCT Filed: Dec. 7, 2022
(86) PCT No.: PCT/US2022/052086

§ 371 Date: Jun. 5, 2024

Related U.S. Application Data
(60) Provisional application No. 63/286,827, filed on Dec. 7, 2021.

(51) Int. Cl. C12N 15/113

(52) U.S. Cl. CPC C12N 15/113; C12N 2310/11; C12N 2310/321; C12N 2310/322; C12N 2310/3231

(57) ABSTRACT

The compositions and methods described herein are related to the identification of disease related genes, the expression of which involves a fraction of transcripts with a retained intron, comprising antisense oligonucleotides (ASO) compositions and methods that target genes which meet two criteria: the genes have a fraction of transcripts that retains an intron, and haploinsufficiency of the gene is associated with disease.

### CROSS-REFERENCE TO RELATED APPLICATIONS

This application claims benefit under 35 U.S.C. § 119 (e) of U.S. Provisional Application No. 63/286,827 filed Dec. 7, 2021, the contents of which are incorporated herein by reference in their entirety.

### TECHNICAL FIELD

The technology described herein relates to the use of antisense oligonucleotides for the upregulation of gene expression.

### BACKGROUND

Antisense oligonucleotides (ASO) were initially investigated for use in down-regulating the expression of selected target genes. However, more recently, ASOs have been shown to actually promote expression of certain genes, e.g., by modifying RNA processing or by suppressing the influence of translation-repressing elements in the genes' transcripts. One such mechanism of upregulation is through targeting an intron retained in a transcript, thereby modifying splicing and/or reducing the abundance of the intron-retaining transcripts, which likely do not produce functional or active protein, or do so less efficiently than fully spliced transcripts. Targeting ASOs to retained intronic sequences can thus increase protein expression. Recently, ASOs targeting retained intronic sequences have been described to increase progranulin levels for the treatment neurodegenerative diseases (WO2020/191212), and the intron-targeting ASO drug nusinersen (Spinraza™, Biogen, Inc.) has been approved for treatment of spinal muscular atrophy (SMA). The identification of additional genes that can be upregulated via ASOs directed to retained introns can provide new approaches for therapeutic intervention.

### SUMMARY

It has been found that a significant number of genes can have a substantial fraction of transcripts that have retained introns relative to the most abundant protein-coding transcripts. The compositions and methods described herein are related to the identification of disease-related genes, the expression of which involves a fraction of transcripts with a retained intron. As described herein, these genes and their transcripts are candidates for increasing expression via ASOs targeting the retained intron sequences. ASOs can thus provide a therapeutic approach for treatment of diseases related to insufficient expression of the products of these genes. In some embodiments, described herein are ASO compositions and methods that target genes which meet two criteria: the genes have a fraction of transcripts that retains an intron, and haploinsufficiency of the gene is associated with disease.

In one aspect, described herein is a method of increasing, in a cell, the expression of a polypeptide from a gene selected from those genes listed in Table 1, the method comprising introducing to the cell an oligonucleotide comprising antisense sequence complementary to a retained intron of a transcript produced by the gene, wherein the oligonucleotide increases expression of the polypeptide in the cell.

In one embodiment of this aspect and all other aspects described herein, the retained intron for the gene's transcript is located at the intron location for that gene set out in Table 1.

In one embodiment of this aspect and all other aspects described herein, antisense oligonucleotides that target a retained intron as described herein do not increase the amount of the target protein by modulating alternative splicing of the pre-mRNA transcript. In one embodiment of this aspect and all other aspects described herein, the antisense oligonucleotides that target a retained intron as described herein do not increase the amount of the target protein by modulating aberrant splicing resulting from mutation of the gene encoding the target protein.

In another embodiment of this aspect and all other aspects described herein, the antisense sequence complementary to a retained intron is at least 8 nucleotides in length. In another embodiment of this and all other aspects described herein, the oligonucleotide is 8 to 40 nucleotides in length.

In another embodiment of this aspect and all other aspects described herein, the oligonucleotide comprises at least one modified sugar moiety or modified linkage.

In another embodiment of this aspect and all other aspects described herein, the oligonucleotide comprises one or more of:
(a) at least one modified nucleotide which comprises a modified sugar moiety;
(b) a 2′ modification of a sugar moiety;
(c) a 2′-O-methyl, 2′-methoxyethoxy, 2′-O-methoxyethyl, 2′-dimethylaminooxyethoxy, 2′-dimethylaminoethoxyethoxy, 2′-fluoro, or 2′-acetamide modification on every sugar moiety;
(d) a locked nucleic acid (LNA) nucleobase; and
(e) at least one modified linkage.

In another embodiment of this aspect and all other aspects described herein, the gene is EHMT1 and the oligonucleotide comprises a sequence selected from SEQ ID Nos: 1-16 or a sequence at least 80% identical thereto.

In another embodiment of this aspect and all other aspects described herein, the gene is EHMT1 and the oligonucleotide has a sequence that consists essentially of a sequence selected from SEQ ID Nos: 1-16 or a sequence at least 80% identical thereto.

In another embodiment of this aspect and all other aspects described herein, the gene is EHMT1 and the oligonucleotide has a sequence that consists of a sequence selected from SEQ ID Nos. 1-16 or a sequence at least 80% identical thereto.

In another embodiment of this aspect and all other aspects described herein, the gene is SIRT6 and the oligonucleotide has a sequence that comprises a sequence selected from SEQ ID Nos: 17-40 or a sequence at least 80% identical thereto.

In another embodiment of this aspect and all other aspects described herein, the gene is SIRT6 and the oligonucleotide has a sequence that consists essentially of a sequence selected from SEQ ID Nos: 17-40 or a sequence at least 80% identical thereto.

In another embodiment of this aspect and all other aspects described herein, the gene is SIRT6 and the oligonucleotide has a sequence that consists of a sequence selected from SEQ ID Nos: 17-40 or a sequence at least 80% identical thereto.

In another embodiment of this aspect and all other aspects described herein, the gene is ENG and the oligonucleotide has a sequence that comprises a sequence selected from SEQ ID Nos: 41-56 or a sequence at least 80% identical thereto.

In another embodiment of this aspect and all other aspects described herein, the gene is ENG and the oligonucleotide has a sequence that consists essentially of a sequence selected from SEQ ID Nos: 41-56 or a sequence at least 80% identical thereto.

In another embodiment of this aspect and all other aspects described herein, the gene is ENG and the oligonucleotide has a sequence that consists of a sequence selected from SEQ ID Nos: 41-56 or a sequence at least 80% identical thereto.

In another embodiment of this aspect and all other aspects described herein, the gene is GCH1 and the oligonucleotide has a sequence that comprises a sequence selected from SEQ ID Nos: 57-64 or a sequence at least 80% identical thereto.

In another embodiment of this aspect and all other aspects described herein, the gene is GCH1 and the oligonucleotide has a sequence that consists essentially of a sequence selected from SEQ ID Nos: 57-64 or a sequence at least 80% identical thereto.

In another embodiment of this aspect and all other aspects described herein, the gene is GCH1 and the oligonucleotide has a sequence that consists of a sequence selected from SEQ ID Nos: 57-64 or a sequence at least 80% identical thereto.

In another embodiment of this aspect and all other aspects described herein, the gene is MEN1 and the oligonucleotide has a sequence that comprises a sequence selected from SEQ ID Nos: 65-72 or a sequence at least 80% identical thereto.

In another embodiment of this aspect and all other aspects described herein, the gene is MEN1 and the oligonucleotide has a sequence that consists essentially of a sequence selected from SEQ ID Nos: 65-72 or a sequence at least 80% identical thereto.

In another embodiment of this aspect and all other aspects described herein, the gene is MEN1 and the oligonucleotide has a sequence that consists of a sequence selected from SEQ ID Nos: 65-72 or a sequence at least 80% identical thereto.

In another embodiment of this aspect and all other aspects described herein, the gene is SON and the oligonucleotide has a sequence that comprises a sequence selected from SEQ ID Nos: 73-74 or a sequence at least 80% identical thereto.

In another embodiment of this aspect and all other aspects described herein, the gene is SON and the oligonucleotide has a sequence that consists essentially of a sequence selected from SEQ ID Nos: 73-74 or a sequence at least 80% identical thereto.

In another embodiment of this aspect and all other aspects described herein, the gene is SON and the oligonucleotide has a sequence that consists of a sequence selected from SEQ ID Nos: 73-74 or a sequence at least 80% identical thereto.

In another embodiment of this aspect and all other aspects described herein, the gene is TSC2 and the oligonucleotide has a sequence that comprises a sequence selected from SEQ ID Nos: 75-92 or a sequence at least 80% identical thereto.

In another embodiment of this aspect and all other aspects described herein, the gene is TSC2 and the oligonucleotide has a sequence that consists essentially of a sequence selected from SEQ ID Nos: 75-92 or a sequence at least 80% identical thereto.

In another embodiment of this aspect and all other aspects described herein, the gene is TSC2 and the oligonucleotide has a sequence that consists of a sequence selected from SEQ ID Nos: 75-92 or a sequence at least 80% identical thereto.

In another embodiment of this aspect and all other aspects described herein, the gene is CDH1 and the oligonucleotide has a sequence that comprises a sequence selected from SEQ ID Nos: 93-100 or a sequence at least 80% identical thereto.

In another embodiment of this aspect and all other aspects described herein, the gene is CDH1 and the oligonucleotide has a sequence that consists essentially of a sequence selected from SEQ ID Nos: 93-100 or a sequence at least 80% identical thereto.

In another embodiment of this aspect and all other aspects described herein, the gene is CDH1 and the oligonucleotide has a sequence that consists of a sequence selected from SEQ ID Nos: 93-100 or a sequence at least 80% identical thereto.

In another embodiment of this aspect and all other aspects described herein, the gene is CAMK2B and the oligonucleotide has a sequence that comprises a sequence selected from SEQ ID Nos: 101-120 or a sequence at least 80% identical thereto.

In another embodiment of this aspect and all other aspects described herein, the gene is CAMK2B and the oligonucleotide has a sequence that consists essentially of a sequence selected from SEQ ID Nos: 101-120 or a sequence at least 80% identical thereto.

In another embodiment of this aspect and all other aspects described herein, the gene is CAMK2B and the oligonucleotide has a sequence that consists of a sequence selected from SEQ ID Nos: 101-120 or a sequence at least 80% identical thereto.

In another embodiment of this aspect and all other aspects described herein, the gene is DNMT3A and the oligonucleotide has a sequence that comprises a sequence selected from SEQ ID Nos: 121-128 or a sequence at least 80% identical thereto.

In another embodiment of this aspect and all other aspects described herein, the gene is DNMT3A and the oligonucleotide has a sequence that consists essentially of a sequence selected from SEQ ID Nos: 121-128 or a sequence at least 80% identical thereto.

In another embodiment of this aspect and all other aspects described herein, the gene is DNMT3A and the oligonucleotide has a sequence that consists of a sequence selected from SEQ ID Nos: 121-128 or a sequence at least 80% identical thereto.

In another embodiment of this aspect and all other aspects described herein, the gene is GIGYF2 and the oligonucleotide has a sequence that comprises a sequence selected from SEQ ID Nos: 129-136 or a sequence at least 80% identical thereto.

In another embodiment of this aspect and all other aspects described herein, the gene is GIGYF2 and the oligonucleotide has a sequence that consists essentially of a sequence selected from SEQ ID Nos: 129-136 or a sequence at least 80% identical thereto.

In another embodiment of this aspect and all other aspects described herein, the gene is GIGYF2 and the oligonucleotide has a sequence that consists of a sequence selected from SEQ ID Nos: 129-136 or a sequence at least 80% identical thereto.

In another embodiment of this aspect and all other aspects described herein, the gene is LMNA and the oligonucleotide has a sequence that comprises a sequence selected from SEQ ID Nos: 137-144 or a sequence at least 80% identical thereto.

In another embodiment of this aspect and all other aspects described herein, the gene is LMNA and the oligonucleotide has a sequence that consists essentially of a sequence selected from SEQ ID Nos: 137-144 or a sequence at least 80% identical thereto.

In another embodiment of this aspect and all other aspects described herein, the gene is LMNA and the oligonucleotide has a sequence that consists of a sequence selected from SEQ ID Nos: 137-144 or a sequence at least 80% identical thereto.

In another embodiment of this aspect and all other aspects described herein, the gene is COL1A1 and the oligonucleotide has a sequence that comprises a sequence selected from SEQ ID Nos: 145-156 or a sequence at least 80% identical thereto.

In another embodiment of this aspect and all other aspects described herein, the gene is COL1A1 and the oligonucleotide has a sequence that consists essentially of a sequence selected from SEQ ID Nos: 145-156 or a sequence at least 80% identical thereto.

In another embodiment of this aspect and all other aspects described herein, the gene is COL1A1 and the oligonucleotide has a sequence that consists of a sequence selected from SEQ ID Nos: 145-156 or a sequence at least 80% identical thereto.

In another embodiment of this aspect and all other aspects described herein, the gene is FH and the oligonucleotide has a sequence that comprises a sequence selected from SEQ ID Nos: 157-164 or a sequence at least 80% identical thereto.

In another embodiment of this aspect and all other aspects described herein, the gene is FH and the oligonucleotide has a sequence that consists essentially of a sequence selected from SEQ ID Nos: 157-164 or a sequence at least 80% identical thereto.

In another embodiment of this aspect and all other aspects described herein, the gene is FH and the oligonucleotide has a sequence that consists of a sequence selected from SEQ ID Nos: 157-164 or a sequence at least 80% identical thereto.

In another embodiment of this aspect and all other aspects described herein, the gene is HMBS and the oligonucleotide has a sequence that comprises a sequence selected from SEQ ID Nos: 165-172 or a sequence at least 80% identical thereto.

In another embodiment of this aspect and all other aspects described herein, the gene is HMBS and the oligonucleotide has a sequence that consists essentially of a sequence selected from SEQ ID Nos: 165-172 or a sequence at least 80% identical thereto.

In another embodiment of this aspect and all other aspects described herein, the gene is HMBS and the oligonucleotide has a sequence that consists of a sequence selected from SEQ ID Nos: 165-172 or a sequence at least 80% identical thereto.

In another embodiment of this aspect and all other aspects described herein, the gene is LEMD3 and the oligonucleotide has a sequence that comprises a sequence selected from SEQ ID Nos: 173-174 or a sequence at least 80% identical thereto.

In another embodiment of this aspect and all other aspects described herein, the gene is LEMD3 and the oligonucleotide has a sequence that consists essentially of a sequence selected from SEQ ID Nos: 173-174 or a sequence at least 80% identical thereto.

In another embodiment of this aspect and all other aspects described herein, the gene is LEMD3 and the oligonucleotide has a sequence that consists of a sequence selected from SEQ ID Nos: 173-174 or a sequence at least 80% identical thereto.

In another embodiment of this aspect and all other aspects described herein, the gene is POLD1 and the oligonucleotide has a sequence that comprises a sequence selected from SEQ ID Nos: 175-194 or a sequence at least 80% identical thereto.

In another embodiment of this aspect and all other aspects described herein, the gene is POLD1 and the oligonucleotide has a sequence that consists essentially of a sequence selected from SEQ ID Nos: 175-194 or a sequence at least 80% identical thereto.

In another embodiment of this aspect and all other aspects described herein, the gene is POLD1 and the oligonucleotide has a sequence that consists of a sequence selected from SEQ ID Nos: 175-194 or a sequence at least 80% identical thereto.

In another embodiment of this aspect and all other aspects described herein, the gene is POLE and the oligonucleotide has a sequence that comprises a sequence selected from SEQ ID Nos: 195-202 or a sequence at least 80% identical thereto.

In another embodiment of this aspect and all other aspects described herein, the gene is POLE and the oligonucleotide has a sequence that consists essentially of a sequence selected from SEQ ID Nos: 195-202 or a sequence at least 80% identical thereto.

In another embodiment of this aspect and all other aspects described herein, the gene is POLE and the oligonucleotide has a sequence that consists of a sequence selected from SEQ ID Nos: 195-202 or a sequence at least 80% identical thereto.

In another embodiment of this aspect and all other aspects described herein, the gene is PPP2R5D and the oligonucleotide has a sequence that comprises a sequence selected from SEQ ID Nos: 203-204 or a sequence at least 80% identical thereto.

In another embodiment of this aspect and all other aspects described herein, the gene is PPP2R5D and the oligonucleotide has a sequence that consists essentially of a sequence selected from SEQ ID Nos: 203-204 or a sequence at least 80% identical thereto.

In another embodiment of this aspect and all other aspects described herein, the gene is PPP2R5D and the oligonucleotide has a sequence that consists of a sequence selected from SEQ ID Nos: 203-204 or a sequence at least 80% identical thereto.

In another embodiment of this aspect and all other aspects described herein, the gene is SF3B4 and the oligonucleotide has a sequence that comprises a sequence selected from SEQ ID Nos: 205-220 or a sequence at least 80% identical thereto.

In another embodiment of this aspect and all other aspects described herein, the gene is SF3B4 and the oligonucleotide has a sequence that consists essentially of a sequence selected from SEQ ID Nos: 205-220 or a sequence at least 80% identical thereto.

In another embodiment of this aspect and all other aspects described herein, the gene is SF3B4 and the oligonucleotide has a sequence that consists of a sequence selected from SEQ ID Nos: 205-220 or a sequence at least 80% identical thereto.

In another embodiment of this aspect and all other aspects described herein, the gene is TDGF1 and the oligonucleotide has a sequence that comprises a sequence selected from SEQ ID Nos: 221-222 or a sequence at least 80% identical thereto.

In another embodiment of this aspect and all other aspects described herein, the gene is TDGF1 and the oligonucleotide has a sequence that consists essentially of a sequence selected from SEQ ID Nos: 221-222 or a sequence at least 80% identical thereto.

In another embodiment of this aspect and all other aspects described herein, the gene is TDGF1 and the oligonucleotide has a sequence that consists of a sequence selected from SEQ ID Nos: 221-222 or a sequence at least 80% identical thereto.

In another embodiment of this aspect and all other aspects described herein, the gene is ZWILCH and the oligonucleotide has a sequence that comprises a sequence selected from SEQ ID Nos: 223-230 or a sequence at least 80% identical thereto.

In another embodiment of this aspect and all other aspects described herein, the gene is ZWILCH and the oligonucleotide has a sequence that consists essentially of a sequence selected from SEQ ID Nos: 223-230 or a sequence at least 80% identical thereto.

In another embodiment of this aspect and all other aspects described herein, the gene is ZWILCH and the oligonucleotide has a sequence that consists of a sequence selected from SEQ ID Nos: 223-230 or a sequence at least 80% identical thereto.

In one embodiment of this aspect and all other aspects described herein, the composition comprises an oligonucleotide at least 80%, 85%, 90%, 91%, 92%, 93%, 94%, 95%, 96%, 97%, 98%, 99% or 100% identical to an oligonucleotide of any one of SEQ ID Nos: 1-230.

In one aspect, described herein is a composition comprising an oligonucleotide of any one of SEQ ID NOS 1-230 or a sequence at least 80% identical thereto, wherein the oligonucleotide comprises at least one modified sugar moiety or modified linkage.

In one embodiment of this aspect and all other aspects described herein, the oligonucleotide comprises one or more of:
(a) at least one modified nucleotide which comprises a modified sugar moiety;
(b) a 2′ modification of a sugar moiety;
(c) a 2′-O-methyl, 2′-methoxyethoxy, 2′-O-methoxyethyl, 2′-dimethylaminooxyethoxy, 2′-dimethylaminoethoxyethoxy, 2′-fluoro, or 2′-acetamide modification on every sugar moiety;
(d) a locked nucleic acid (LNA) nucleobase; and (e) at least one modified linkage.

In one embodiment of this aspect and all other aspects described herein, the pharmaceutical composition comprises a pharmaceutically acceptable carrier.

In one aspect, described herein is a method of treating a disease or disorder associated with insufficient expression of EHMT1 in a subject in need thereof, the method comprising administering a composition comprising an antisense oligonucleotide selected from SEQ ID NOS: 1-16 or a sequence at least 80% identical thereto to the subject, whereby expression of EHMT1 is increased, thereby treating the disease or disorder.

In one aspect, described herein is a method of treating Kleefstra syndrome in a subject in need thereof, the method comprising administering a composition comprising an antisense oligonucleotide selected from SEQ ID NOS: 1-16 or a sequence at least 80% identical thereto to the subject, whereby expression of EHMT1 is increased, thereby treating Kleefstra syndrome.

In one aspect, described herein is a method of treating a disease or disorder associated with insufficient expression of SIRT6 in a subject in need thereof, the method comprising administering a composition comprising an antisense oligonucleotide selected from SEQ ID NOS 17-40 or a sequence at least 80% identical thereto to the subject, whereby expression of SIRT6 is increased, thereby treating the disease or disorder.

In one aspect, described herein is a method of treating Alzheimer's disease in a subject in need thereof, the method comprising administering a composition comprising an antisense oligonucleotide selected from SEQ ID NOS 17-40 or a sequence at least 80% identical thereto to the subject, whereby expression of SIRT6 is increased, thereby treating Alzheimer's disease.

In one aspect, described herein is a method of treating a disease or disorder associated with insufficient expression of ENG in a subject in need thereof, the method comprising administering a composition comprising an antisense oligonucleotide selected from SEQ ID NOS 41-56 or a sequence at least 80% identical thereto to the subject, whereby expression of ENG is increased, thereby treating the disease or disorder.

In one aspect, described herein is a method of treating hereditary hemorrhagic telangiectasia in a subject in need thereof, the method comprising administering a composition comprising an antisense oligonucleotide selected from SEQ ID NOS 41-56 or a sequence at least 80% identical thereto to the subject, whereby expression of ENG is increased, thereby treating Hereditary Hemorrhagic Telangiectasia.

In one aspect, described herein is a method of treating a disease or disorder associated with insufficient expression of GCH1 in a subject in need thereof, the method comprising administering a composition comprising an antisense oligonucleotide selected from SEQ ID NOS 57-64 or a sequence at least 80% identical thereto to the subject, whereby expression of GCH1 is increased, thereby treating the disease or disorder.

In one aspect, described herein is a method of treating Dopa-responsive dystonia in a subject in need thereof, the method comprising administering a composition comprising an antisense oligonucleotide selected from SEQ ID NOS 57-64 or a sequence at least 80% identical thereto to the subject, whereby expression of GCH1 is increased, thereby treating Dopa-responsive dystonia.

In one aspect, described herein is a method of treating a disease or disorder associated with insufficient expression of MEN1 in a subject in need thereof, the method comprising administering a composition comprising an antisense oligonucleotide selected from SEQ ID NOS 65-72 or a sequence at least 80% identical thereto to the subject, whereby expression of MEN1 is increased, thereby treating the disease or disorder.

In one aspect, described herein is a method of treating multiple endocrine neoplasia type 1 in a subject in need thereof, the method comprising administering a composition comprising an antisense oligonucleotide selected from SEQ ID NOS 65-72 or a sequence at least 80% identical thereto to the subject, whereby expression of MEN1 is increased, thereby treating Multiple endocrine neoplasia type 1.

In one aspect, described herein is a method of treating a disease or disorder associated with insufficient expression of SON in a subject in need thereof, the method comprising administering a composition comprising an antisense oligonucleotide selected from SEQ ID NOS 73-74 or a sequence at least 80% identical thereto to the subject, whereby expression of SON is increased, thereby treating the disease or disorder.

In one aspect, described herein is a method of treating ZTTK syndrome in a subject in need thereof, the method comprising administering a composition comprising an antisense oligonucleotide selected from SEQ ID NOS 73-74 or a sequence at least 80% identical thereto to the subject, whereby expression of SON is increased, thereby treating ZTTK syndrome.

In one aspect, described herein is a method of treating a disease or disorder associated with insufficient expression of TSC2 in a subject in need thereof, the method comprising administering a composition comprising an antisense oligonucleotide selected from SEQ ID NOS 75-92 or a sequence at least 80% identical thereto to the subject, whereby expression of TSC2 is increased, thereby treating the disease or disorder.

In one aspect, described herein is a method of treating tuberous sclerosis 2 in a subject in need thereof, the method comprising administering a composition comprising an antisense oligonucleotide selected from SEQ ID NOS 75-92 or a sequence at least 80% identical thereto to the subject, whereby expression of TSC2 is increased, thereby treating tuberous sclerosis 2.

In one aspect, described herein is a method of treating a disease or disorder associated with insufficient expression of CDH1 in a subject in need thereof, the method comprising administering a composition comprising an antisense oligonucleotide selected from SEQ ID NOS 93-100 or a sequence at least 80% identical thereto to the subject, whereby expression of CDH1 is increased, thereby treating the disease or disorder.

In one aspect, described herein is a method of treating hereditary diffuse gastric adenocarcinoma in a subject in need thereof, the method comprising administering a composition comprising an antisense oligonucleotide selected from SEQ ID NOS 93-100 or a sequence at least 80% identical thereto to the subject, whereby expression of CDH1 is increased, thereby treating hereditary diffuse gastric adenocarcinoma.

In one aspect, described herein is a method of treating a disease or disorder associated with insufficient expression of CAMK2B, in a subject in need thereof. The method comprises administering a composition comprising an antisense oligonucleotide selected from SEQ ID NOS 101-120 or a sequence at least 80% identical thereto to the subject, whereby expression of CAMK2B is increased, thereby treating the disease or disorder.

In one aspect, described herein is a method of treating intellectual developmental disorder, such as intellectual developmental disorder associated with insufficient expression of CAMK2B, in a subject in need thereof. The method comprises administering a composition comprising an antisense oligonucleotide selected from SEQ ID NOS 101-120 or a sequence at least 80% identical thereto to the subject, whereby expression of CAMK2B is increased, thereby treating the intellectual developmental disorder.

In one aspect, described herein is a method of treating a disease or disorder associated with insufficient expression of DNMT3 in a subject in need thereof, the method comprising administering a composition comprising an antisense oligonucleotide selected from SEQ ID NOS 121-128 or a sequence at least 80% identical thereto to the subject, whereby expression of DNMT3 is increased, thereby treating the disease or disorder.

In one aspect, described herein is a method of treating Tattan-Brown-Rahman syndrome in a subject in need thereof, the method comprising administering a composition comprising an antisense oligonucleotide selected from SEQ ID NOS 121-128 or a sequence at least 80% identical thereto to the subject, whereby expression of DNMT3 is increased, thereby treating Tattan-Brown-Rahman syndrome.

In one aspect, described herein is a method of treating a disease or disorder associated with insufficient expression of GIGYF2 in a subject in need thereof, the method comprising administering a composition comprising an antisense oligonucleotide selected from SEQ ID NOS 129-136 or a sequence at least 80% identical thereto to the subject, whereby expression of GIGYF2 is increased, thereby treating the disease or disorder.

In one aspect, described herein is a method of treating a neurodevelopmental disorder associated with insufficient expression of GIGYF2 in a subject in need thereof, the method comprising administering a composition comprising an antisense oligonucleotide selected from SEQ ID NOS 129-136 or a sequence at least 80% identical thereto to the subject, whereby expression of GIGYF2 is increased, thereby treating the neurodevelopmental disorder.

In one aspect, described herein is a method of treating a disease or disorder associated with insufficient expression of LMNA in a subject in need thereof, the method comprising administering a composition comprising an antisense oligonucleotide selected from SEQ ID NOS 137-144 or a sequence at least 80% identical thereto to the subject, whereby expression of LMNA is increased, thereby treating the disease or disorder.

In one aspect, described herein is a method of treating dilated cardiomyopathy in a subject in need thereof, the method comprising administering a composition comprising an antisense oligonucleotide selected from SEQ ID NOS 137-144 or a sequence at least 80% identical thereto to the subject, whereby expression of LMNA is increased, thereby treating dilated cardiomyopathy.

In one aspect, described herein is a method of treating a disease or disorder associated with insufficient expression of COL1A1 in a subject in need thereof, the method comprising administering a composition comprising an antisense oligonucleotide selected from SEQ ID NOS 145-156 or a sequence at least 80% identical thereto to the subject, whereby expression of COL1A1 is increased, thereby treating the disease or disorder.

In one aspect, described herein is a method of treating osteogenesis imperfecta type 1 in a subject in need thereof, the method comprising administering a composition comprising an antisense oligonucleotide selected from SEQ ID NOS 145-156 or a sequence at least 80% identical thereto to the subject, whereby expression of COL1A1 is increased, thereby treating osteogenesis imperfecta type 1.

In one aspect, described herein is a method of treating a disease or disorder associated with insufficient expression of FH in a subject in need thereof, the method comprising administering a composition comprising an antisense oligonucleotide selected from SEQ ID NOS 157-164 or a sequence at least 80% identical thereto to the subject, whereby expression of FH is increased, thereby treating the disease or disorder.

In one aspect, described herein is a method of treating hereditary leiomyomatosis renal cancer in a subject in need thereof, the method comprising administering a composition comprising an antisense oligonucleotide selected from SEQ ID NOS 157-164 or a sequence at least 80% identical thereto to the subject, whereby expression of FH is increased, thereby treating Hereditary leiomyomatosis.

In one aspect, described herein is a method of treating a disease or disorder associated with insufficient expression of HMBS in a subject in need thereof, the method comprising administering a composition comprising an antisense oligonucleotide selected from SEQ ID NOS 165-172 or a sequence at least 80% identical thereto to the subject, whereby expression of HMBS is increased, thereby treating the disease or disorder.

In one aspect, described herein is a method of treating acute intermittent porphyria in a subject in need thereof, the method comprising administering a composition comprising an antisense oligonucleotide selected from SEQ ID NOS 165-172 or a sequence at least 80% identical thereto to the subject, whereby expression of HMBS is increased, thereby treating Acute intermittent porphyria.

In one aspect, described herein is a method of treating a disease or disorder associated with insufficient expression of LEMD in a subject in need thereof, the method comprising administering a composition comprising an antisense oligonucleotide selected from SEQ ID NOS 173-174 or a sequence at least 80% identical thereto to the subject, whereby expression of LEMD is increased, thereby treating the disease or disorder.

In one aspect, described herein is a method of treating Buschke-Ollendorff syndrome in a subject in need thereof, the method comprising administering a composition comprising an antisense oligonucleotide selected from SEQ ID NOS 173-174 or a sequence at least 80% identical thereto to the subject, whereby expression of LEMD is increased, thereby treating Buschke-ollendorff syndrome.

In one aspect, described herein is a method of treating a disease or disorder associated with insufficient expression of POLD1 in a subject in need thereof, the method comprising administering a composition comprising an antisense oligonucleotide selected from SEQ ID NOS 175-194 or a sequence at least 80% identical thereto to the subject, whereby expression of POLD1 is increased, thereby treating the disease or disorder.

In one aspect, described herein is a method of treating early onset colon cancer in a subject in need thereof, the method comprising administering a composition comprising an antisense oligonucleotide selected from SEQ ID NOS 175-194 or a sequence at least 80% identical thereto to the subject, whereby expression of POLD1 is increased, thereby treating Early onset colon cancer.

In one aspect, described herein is a method of treating a disease or disorder associated with insufficient expression of POLE in a subject in need thereof, the method comprising administering a composition comprising an antisense oligonucleotide selected from SEQ ID NOS 195-202 or a sequence at least 80% identical thereto to the subject, whereby expression of POLE is increased, thereby treating the disease or disorder.

In one aspect, described herein is a method of treating colon polyposis in a subject in need thereof, the method comprising administering a composition comprising an antisense oligonucleotide selected from SEQ ID NOS 195-202 or a sequence at least 80% identical thereto to the subject, whereby expression of POLE is increased, thereby treating Colon polyposis.

In one aspect, described herein is a method of treating a disease or disorder associated with insufficient expression of PPP2R5D in a subject in need thereof, the method comprising administering a composition comprising an antisense oligonucleotide selected from SEQ ID NOS 203-204 or a sequence at least 80% identical thereto to the subject, whereby expression of PPP2R5D is increased, thereby treating the disease or disorder.

In one aspect, described herein is a method of treating a neurodevelopmental disorder associated with insufficient expression of PPP2R5D in a subject in need thereof, the method comprising administering a composition comprising an antisense oligonucleotide selected from SEQ ID NOS 203-204 or a sequence at least 80% identical thereto to the subject, whereby expression of PPP2R5D is increased, thereby treating the neurodevelopmental disorder.

In one aspect, described herein is a method of treating a disease or disorder associated with insufficient expression of TDGF1 in a subject in need thereof, the method comprising administering a composition comprising an antisense oligonucleotide selected from SEQ ID NOS 221-222 or a sequence at least 80% identical thereto to the subject, whereby expression of TDGF1 is increased, thereby treating the disease or disorder.

In one aspect, described herein is a method of treating a neurodevelopmental disorders associated with insufficient expression of TDGF1 in a subject in need thereof, the method comprising administering a composition comprising an antisense oligonucleotide selected from SEQ ID NOS 221-222 or a sequence at least 80% identical thereto to the subject, whereby expression of TDGF1 is increased, thereby treating the neurodevelopmental disorders.

In one aspect, described herein is a method of treating a disease or disorder associated with insufficient expression of SF3B4 in a subject in need thereof, the method comprising administering a composition comprising an antisense oligonucleotide selected from SEQ ID NOS 205-220 or a sequence at least 80% identical thereto to the subject, whereby expression of SF3B4 is increased, thereby treating the disease or disorder.

In one aspect, described herein is a method of treating Nager syndrome in a subject in need thereof, the method comprising administering a composition comprising an antisense oligonucleotide selected from SEQ ID NOS 205-220 or a sequence at least 80% identical thereto to the subject, whereby expression of SF3B4 is increased, thereby treating Nager syndrome.

In one aspect, described herein is a method of treating a disease or disorder associated with insufficient expression of ZWILCH in a subject in need thereof, the method comprising administering a composition comprising an antisense oligonucleotide selected from SEQ ID NOS 223-230 or a sequence at least 80% identical thereto to the subject, whereby expression of ZWILCH is increased, thereby treating the disease or disorder.

In one aspect, described herein is a method of treating autism in a subject in need thereof, the method comprising administering a composition comprising an antisense oligonucleotide selected from SEQ ID NOS 223-230 or a sequence at least 80% identical thereto to the subject, whereby expression of ZWILCH is increased, thereby treating autism.

In one embodiment of this aspect and all other aspects described herein, the antisense oligonucleotide comprises at least one modified sugar moiety or modified linkage.

In one embodiment of this aspect and all other aspects described herein, the oligonucleotide comprises at least one of:
(a) at least one modified nucleotide which comprises a modified sugar moiety;
(b) a 2′ modification of a sugar moiety;
(c) a 2′-O-methyl, 2′-methoxyethoxy, 2′-O-methoxyethyl, 2′-dimethylaminooxyethoxy, 2′-dimethylaminoethoxyethoxy, 2′-fluoro, or 2′-acetamide modification on every sugar moiety; and

(d) a locked nucleic acid (LNA) nucleobase; and

(e) at least one modified linkage.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The terms “increased”, “increase”, “enhance”, “activate” are all used herein to refer to an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level

The term “improve” or “improvement,” when applied to a score in a standardized scale or rating, e.g., for disease symptoms or severity, means a statistically significant, favorable change in the scale or rating on that scale.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to a reference level, for example, a decrease of at least about 20%, or at least about 30%, or at least about 40%, or at least about 50%, or at least about 60%, or at least about 70%, or at least about 80%, or at least about 90%, or at least about 95% as compared to a reference level. As used herein, “reduction” or “inhibition” does not encompass a complete inhibition or reduction as compared to a reference level. “Complete inhibition” is a 100% inhibition as compared to a reference level.

As used herein, a “reference level” refers to the level or value for a given parameter against which one compares the level or value in a given sample or situation to determine whether the level or value has changed in a meaningful way. A reference level can be a level in or from a sample that is not treated to change the parameter. A reference level can alternatively be a level in or from a normal or otherwise unaffected sample. A reference level can alternatively be a level in or from a sample obtained from a subject at a prior time point, for example, prior to a given treatment.

As used herein, an “appropriate control” refers to an untreated, otherwise identical cell or population (e.g., a subject who was not administered an agent described herein, or was administered by only a subset of agents described herein, as compared to a non-control cell).

The term “modulation,” when applied to target gene expression refers to stimulation; i.e., an increase in expression. This modulation can be measured in ways which are routine in the art, for example by Northern blot assay, RNase protection assay or reverse transcriptase PCR for measurement of transcription or splicing products or mRNA, or by Western blot, ELISA or immunoprecipitation assay for protein expression. Effects of antisense oligonucleotides as described herein on target gene expression can also be determined as taught in the examples herein or, for example, in WO2020/191212, which is incorporated herein by reference.

As used herein, an “exon” refers to any part of a primary gene transcript that is comprised by the final mature RNA produced by that gene after introns have been removed by RNA splicing. The term exon refers to both the DNA sequence within a gene and to the corresponding sequence in RNA transcripts.

As used herein, an “intron” refers to any nucleotide sequence within a gene or primary transcript of the gene that is removed by RNA splicing during maturation of the final RNA product. The term intron refers to both the DNA sequence within a gene and the corresponding sequence in RNA transcripts.

As used herein, the term “alternative splicing” refers to a regulated process during gene expression that results in a single gene coding for more than one protein. In this process, particular exons of a gene may be included within or excluded from the final, processed messenger RNA (mRNA) produced from that gene. In one embodiment, the compositions and methods described herein do not modulate alternative splicing.

As used herein, the term “retained intron” refers to an intron sequence that is not spliced out of a given spliced RNA transcript. As discussed further elsewhere herein, intron retention can occur in a fraction of spliced transcripts for a given gene, and results in a transcript that is not efficiently translated to produce a functional protein product. The fraction of intron retention can vary with the gene, or for example, the tissue- or cell type, but any level of retention detectable by the methods described herein can be a fraction retained (FR) or retained fraction.

The term “therapeutically effective amount” refers to an amount of an ASO or pharmaceutical composition comprising an ASO as described herein that is sufficient to provide a particular beneficial effect when administered to a typical subject. An effective amount as used herein would also include an amount sufficient to delay the development of a symptom of a disease, alter the course of a symptom of a disease (for example but not limited to, slow the progression of a symptom of a disease), or reverse a symptom of a disease. Thus, it is not possible to specify an exact “effective amount” for every situation; however, for any given case, an appropriate “effective amount” can be determined by one of ordinary skill in the art using only routine experimentation.

In one embodiment, a composition as described herein, e.g., a pharmaceutical composition or formulation, further comprises a pharmaceutically acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an agent. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

As used herein, a “subject” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of diseases. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, a “subject in need” of treatment for a particular condition can be a subject having that condition, diagnosed as having that condition, or at increased risk of developing that condition.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIG. 1 shows a visualization of Intron Retention (IR) from analysis of Iso-Seq data for GRN with explanatory labels. The fraction of IR transcripts is computed as 51/(2+2+2+2+3+3+3+3+3+4+51+137)=51/215=0.24 (alternatively, 24%).

FIG. 2 shows an ASO design methodology example. The intronic splice suppression potential of hexamer motifs tiled across the target intron is evaluated. Regions with high average scores are identified (shaded) for targeting with ASOs.

### DETAILED DESCRIPTION

Where disease symptoms result from an insufficient amount of a given protein, strategies for increasing the amount of such protein can be explored as treatment options. The methods and compositions described herein are based, in part, on the discovery that genes can have a substantial fraction of transcripts that have retained introns relative to the fully-spliced protein-coding transcripts. These intron-retaining transcripts likely do not produce active or functional protein, or do so less efficiently, and subsequently can have repressive effects on the levels of functional protein produced. Targeting these intron-retraining transcripts can serve as an effective means of boosting functional protein levels and thereby treating diseases or disorders associated with insufficient levels of such proteins.

Antisense oligonucleotides (ASOs) have been widely studied for the down-regulation of gene expression, but more recently ASOs have been used to target intronic regions to increase production of proteins.

As described herein, a systematic evaluation of genes that produce a measurable fraction of transcripts with a retained intron identified a number of genes associated with disease resulting from or related to insufficient expression of those genes. Where the targeting of retained introns with ASOs can promote increased levels of the encoded proteins, each of these genes, and their associated diseases, becomes an attractive candidate for treatment using ASOs. While any gene which produces transcripts with retained introns can be a target for modulation, genes for which haploinsufficiency leads to or is associated with disease are particularly attractive targets for modulation. In haploinsufficiency, the subject has a first allele encoding a functional target protein, and a second allele from which the target protein is not produced, or a second allele encoding a nonfunctional target protein. When the functioning allele does not produce enough functional protein to satisfy demand, symptoms of disease related to the deficiency can result. While it has been found herein that the expression of many genes involves some level of intron retention, those for which haploinsufficiency is associated with disease are of particular interest, because a reduction in the fraction of transcripts with a retained intron can promote expression of functional protein without necessarily needing to correct the defect in the non-functional allele.

The following describes the identification of genes that can be targeted for increased expression by ASOs targeting retained intron sequences, and treatment of diseases related to insufficient expression of those genes. The methods described herein can, for example, reduce the fraction of target transcripts with a retained intron and/or promote splicing to remove retained intron sequence in a target transcript. With this in mind, the following describes target genes and considerations involved in increasing their expression, as well as treating associated disease using ASOs.

### Retained Introns

A characteristic of eukaryotic gene expression is that many genes are transcribed as a primary RNA transcript including protein coding sequences that are interrupted by non-protein coding sequences termed “introns.” Before the protein can be translated from the RNA, the introns must be removed by RNA splicing, which excises the introns and joins the remaining sequences, referred to as “exons”, together. Thus, an “intron” is any nucleotide sequence within a gene that is typically removed by RNA splicing during maturation of the final mRNA product. For any given gene, there can be a plurality of exons and a plurality of introns, with each intron needing to be spliced out before a fully processed mRNA transcript is generated. Among other things, the splicing process can provide an added level of control or flexibility in gene expression, in that some proteins are encoded on transcripts in which so-called alternative splicing permits a single primary transcript to encode more than one protein, depending upon which of two or more alternative exons are included in the final transcript mRNA. As but one example, alternative splicing can generate different versions of a given protein in a tissue- or cell type-specific manner where the same primary transcript is spliced in one way in a first cell type and in another way in a second cell type based on the activity of, for example, different splicing factors or regulators in the different cell types.

It has been recognized, for example, in the survival motor neuron-2 (SMN2) gene (see, e.g., Harahap et al., Brain & Dev. 40:670-677 (2018)}, and in the progranulin (GRN) gene (see, e.g., WO2020/191212), among others, that there can be a fraction of RNA transcripts that retain one or more introns after splicing, and that intron-retaining transcripts are not efficiently translated to functional protein. As discussed above, it is this intron-containing fraction of transcripts for a given gene that provides a target for increasing protein expression via ASOs.

### Identifying Targets for ASO Therapy

A question raised by the discovery of particular intron-retaining transcripts, and the demonstrated ability to up-regulate protein expression by targeting them with ASOs, is whether there are other genes affected by intron retention (IR) that could be similarly amenable to ASO therapy. It was reasoned such a gene should meet the following criteria:
1) a fraction of transcripts retains exactly one intron relative to the canonical protein-coding transcript model—this is referred to as the fraction retained (FR); and
2) haploinsufficiency of the gene is associated with disease. In principle, FR determines the potential of an ASO to boost protein expression. In the analysis described in the Examples herein, transcripts that had any detectable level of FR were considered.

RNA sequence database analyses were performed to identify genes satisfying the criteria set out above, focusing on data from an Alzheimer's brain database, as initial proof of principle—see Example 1 herein below. The same approach can be used to identify genes with FR in other cell types or tissues. It is considered that for a given gene, FR may be different in different tissues or cell types.

Table 1 includes a set of genes identified using the approach set out herein that satisfy the criteria described for genes suitable for therapeutic intervention using ASOs to promote expression. Also provided in Table 1 are, among other things, the human chromosomal locations of the retained intron in each gene, the fraction of transcripts containing the retained intron (FR) for each gene, and the disease or diseases associated with haploinsufficiency. The genome coordinates reported herein are all based on human Genome Reference Consortium Build 38 (GRCh38).

Once genes for which transcripts satisfying both criteria are identified, ASOs can be designed, for example, as exemplified for GRN in WO2020/191212, which is incorporated herein by reference in its entirety. Retained intron sequences can be analyzed for sequence containing or enriched for motifs predicted to repress splicing—see, e.g., Rosenberg, A., et. al. (2015), Learning the Sequence Determinant of Alternative Splicing from Millions of Random Sequences. Cell, 163 (3), 698-711, the entire content of which is incorporated herein by reference. In Rosenberg et. al., the splicing patterns of millions of synthetic minigenes with variable intronic sequences were described. Certain hexamers were found to be enriched in introns which were spliced out with lower efficiency, and more broadly intronic motifs were found to interact additively to regulate the overall level of intron retention. Rosenberg et. al. calculated a score describing the tendency of each hexamer motif to cause retention or splicing-out of minigene introns. This model was applied to intronic sequences in genes showing intron retention, and identified regions with the highest average score as ASO targets. Thus, hexamer motifs tiled across the whole retained intron are evaluated for intronic splice suppression scores, and regions of the retained intron in which hexamer motifs have the highest average score identified as candidate targets for ASOs. These sequences can be tiled with, for example, 19-mer ASOs, which can then be evaluated for activity in promoting target protein expression, e.g., in a suitable in vitro cell culture model, and/or in an in vivo animal model. Table 2 provides candidate ASOs identified in this manner for each gene target listed in Table 1.

### Target Genes for ASO-Mediated Promotion of Expression

Genes set out herein as targets for ASO-mediated promotion of expression are discussed below. The expression of the target genes can be evaluated before and after administration of ASOs, e.g., via immunoassay, including, but not limited to, for example, Western blotting. The effectiveness of a given ASO or ASO preparation or formulation for promoting target gene expression can also be evaluated by measuring the level of retained-intron-containing RNA before and after administration or contacting with the ASO—this can be performed for example, by RNAse protection assay (see, e.g., U.S. Pat. No. 9,976,143) or via RT-PCR (see, e.g., WO2020/191212).

EHMT1 codes for the enzyme euchromatic histone methyltransferase 1, which modifies histone proteins, methylating the lysine-9 position of histone H3 to suppress the activity of certain genes. The sequence of EHMT1 is known for a number of species, e.g., human EHMT1 (NCBI Gene ID: 79813) mRNA (e.g., NM_024757.5) and polypeptide (e.g., NP_079033.4), for example for isoform 1. Antibodies for specific detection of EHMT1 are available, for example, from Abcam, see Anti-EHMT1/GLP antibody ab241306. Candidate ASOs for promoting EHMT1 expression are presented in Table 2, and include SEQ ID Nos. 1-16. Haploinsufficiency of the EHMT1 gene can cause Kleefstra syndrome 1, a rare disease characterized by intellectual developmental delay, mental retardation, psychomotor developmental delay, seizures, behavioral abnormalities, and facial dysmorphisms. Early boosting of EHMT1 is contemplated for alleviating developmental disabilities or other sequelae of EHMT1 deficiency. Developmental aspects can be evaluated, for example, through cognitive test scores, evaluation of seizure activity or neurological activity or evaluation of one or more of the other disease symptoms associated with insufficient expression of EHMT1.

SIRT6 or Sirtuin 6 codes for a member of the sirtuin family of NAD-dependent enzymes, which exhibits ADP-ribosyl transferase and histone deacetylase activities, and plays a role in DNA repair, maintenance of telomeric chromatin, inflammation, lipid and glucose metabolism. SIRT6 sequence is known for a number of species, e.g., human SIRT6 (NCBI Gene ID: 51548) mRNA (e.g., NM_016539.4) and polypeptide (e.g., NP_057623.2), for example for isoform 1. Antibodies for specific detection of SIRT6 are available, for example, from Abcam, see Anti-SIRT6 antibody [EPR18463], ab191385. Candidate ASOs for promoting SIRT6 expression are presented in Table 2, and include SEQ ID Nos. 17-40. Mutations, down-regulation in the brain and haploinsufficiency of the SIRT6 are associated with, for example, neurodegenerative disease, including Alzheimer's disease, making it a potential therapeutic target. Increasing SIRT6 can prevent Aβ42-induced DNA damage and reduce or prevent neurodegeneration or other sequelae of SIRT6 deficiency. Neurodegeneration can be measured, for example, by structural and functional MRIs and/or standardized cognitive testing.

ENG codes for a homodimeric transmembrane protein that is a major glycoprotein of the vascular endothelium and is a component of the transforming growth factor beta receptor complex. ENG or Endoglin sequence is known for a number of species, e.g., human ENG (NCBI Gene ID: 2022) mRNA (e.g., NM_001114753.3) and polypeptide (e.g., NP_001108225.1), for example for isoform 1. Antibodies for specific detection of ENG are available, for example, from R&D Systems—see AF1097. Candidate ASOs for promoting ENG expression are presented in Table 2, and include SEQ ID Nos. 41-56. Mutations and haploinsufficiency of ENG can cause hereditary hemorrhagic telangiectasia, also known as Osler-Rendu-Weber syndrome 1, a disorder that results in the development of multiple abnormalities in blood vessels, potentially resulting in bleeding and shunting of blood. Boosting expression is specifically contemplated for the treatment of Osler-Rendu-Weber syndrome 1 or other sequelae of ENG deficiency. Nosebleeds are often a first sign and can be used as one readout for effectiveness of a treatment, as can changes in iron-deficiency anemia related to the disease.

GCH1 or GTP cyclohydrolase 1 codes for a member of the GTP cyclohydrolase family that is the first and rate-limiting enzyme in tetrahydrobiopterin biosynthesis. GCH1 sequence is known for a number of species, e.g., human GCH1 (NCBI Gene ID: 2643) mRNA, (e.g., NM_000161.3) and polypeptide (e.g., NP_000152.1), for example for isoform 1. Antibodies for specific detection of GCH1 are available, for example, from Invitrogen—see GCH1 Monoclonal antibody OTI3C9. Candidate ASOs for promoting GCH1 expression are presented in Table 2, and include SEQ ID Nos. 57-64. Mutations and haploinsufficiency of GCH1 are associated with dopa-responsive dystonia, a disorder defined by sustained involuntary muscle contractions, often leading to abnormal postures. Boosting GCH1 expression is specifically contemplated to lead to improvement in motor function or improvement in other sequelae of GCH1 deficiency. Motor function can be measured with tensile exercises or standardized evaluations of motor function.

MEN1 codes for menin, a scaffold protein that functions in histone modification and epigenetic gene regulation. MEN1 sequence is known for a number of species, e.g., human MEN1 (NCBI Gene ID: 4221) mRNA (e.g., NM_000244.3) and polypeptide (e.g., NP_000235.2), for example for isoform 1. Antibodies for specific detection of MEN1 are available, for example, from Abcam—see Anti-Menin antibody [EPR3986], ab92443. Candidate ASOs for promoting MEN1 expression are presented in Table 2, and include SEQ ID Nos. 65-72. Mutations and haploinsufficiency are associated with multiple endocrine neoplasia type 1, a disorder characterized by tumors of the parathyroid glands, gastro-intestinal endocrine tissue, the anterior pituitary and other tissues. Boosting MEN1 expression is specifically contemplated to suppress tumors and/or limit or prevent continued tumor growth or other sequelae of MEN1 deficiency.

SON codes for a protein that binds RNA and promotes pre-mRNA splicing, particularly of transcripts with poor splice sites. SON or SON DNA and RNA binding protein sequence is known for a number of species, e.g., human SON (NCBI Gene ID: 6651) mRNA (e.g., NM_001291411.2) and polypeptide (e.g., NP_001278340.2), for example for isoform E. Antibodies for specific detection of SON are available, for example, from Novus—see SON antibody NBP1-88706. Candidate ASOs for promoting SON expression are presented in Table 2, and include SEQ ID Nos. 73-74. Mutations and haploinsufficiency are associated with ZTTK syndrome, a rare genetic, multiple congenital anomalies/dysmorphic syndrome characterized by intellectual disability, developmental delay, epilepsy, vision problems, and congenital malformations. Boosting SON expression is specifically contemplated for alleviating or improving one or more of these symptoms or other sequelae of SON deficiency, which can be measured, for example, by standardized cognitive testing, measurement of the severity or frequency of epileptic events, visual acuity testing or other evaluations of ZTTK symptomatic change.

TSC2 or TSC complex subunit 2 codes for the protein tuberin, the function of which, while not fully understood, seems to be involved in controlling cell growth and size. TSC2 sequence is known for a number of species, e.g., human TSC2 (NCBI Gene ID: 7249) mRNA (e.g., NM_000548.5) and polypeptide (e.g., NP_000539.2), for example for isoform 1. Antibodies for specific detection of TSC2 are available, for example, from R&D Systems—see human/mouse TSC2 antibody MAB40401. Candidate ASOs for promoting TSC2 expression are presented in Table 2, and include SEQ ID Nos. 75-92. Mutations and haploinsufficiency leads to tuberous sclerosis 2, a multi-system disorder that affects the brain, kidneys, heart, and skin, and is characterized by hamartomas and hamartias (benign tumors), as well as epilepsy, learning difficulties, and skin lesions. Boosting TSC2 is specifically contemplated to suppress and/or prevent tumor growth and/or reduce or prevent more severe symptoms including seizures or other sequelae of TSC2 deficiency. Decreased incidence, growth or size of hamartoma or hamartia tissue can provide a measure of treatment efficacy. Evaluations of neural activity or seizure activity can provide a measurement for reduced susceptibility to seizures upon treatment, as can evaluation of improvements in other symptoms or parameters as appropriate.

CDH1 or Cadherin-1 codes for cadherin, a calcium-dependent cell-cell adhesion protein. CDH1 sequence is known for a number of species, e.g., human CDH1 (NCBI Gene ID: 999) mRNA (e.g., NM_001317184.2) and polypeptide (e.g., NP_001304113.1), for example for isoform 2. Antibodies for specific detection of CDH1 are available, for example, from R&D Systems—see human/mouse E-Cadherin antibody AF748. Candidate ASOs for promoting CDH1 expression are presented in Table 2, and include SEQ ID Nos. 93-100. Mutations or haploinsufficiency can lead to hereditary diffuse gastric adenocarcinoma, a cancer predisposition syndrome with increased susceptibility to malignant tumors in the stomach. Boosting CDH1 is specifically contemplated to suppress and/or prevent tumor growth or other sequelae of CDH1 deficiency. Decreased tumor incidence, growth, or size can provide a measure of treatment efficacy as can evaluation of improvements in other symptoms or parameters as appropriate.

CAMK2B or Calcium/calmodulin dependent protein kinase II beta codes for the beta chain of an enzyme that is a member of the serine/threonine protein kinase family, which is important for dendritic spine and synapse formation and neuronal plasticity. CAMK2B sequence is known for a number of species, e.g., human CAMK2B (NCBI Gene ID: 816) mRNA (e.g., NM_001220.5) and polypeptide (e.g., NP_001211.3), for example for isoform 1. Antibodies for specific detection of CAMK2B are available, for example, from Novusbio—see CAMKII beta antibody NB120-22131. Candidate ASOs for promoting CAMK2B expression are presented in Table 2, and include SEQ ID Nos. 101-120. Mutations and haploinsufficiency of CAMK2B can lead to intellectual developmental disorder. Boosting CAMK2B expression is specifically contemplated for the treatment of such disorder or other sequelae of CAMK2B deficiency. Treatment efficacy can be measured, for example, using standardized cognitive testing or evaluation of improvements in other symptoms or parameters as appropriate.

DNMT3A codes for DNA methyltransferase 3 alpha, an enzyme that functions in de novo methylation to regulate gene silencing. DNMT3A sequence is known for a number of species, e.g., human DNMT3A (NCBI Gene ID: 1788) mRNA (e.g., NM_001320892.2) and polypeptide (e.g., NP_001307821.1), for example for isoform c. Antibodies for specific detection of DNMT3A are available, for example, from Abcam—see Anti-Dnmt3a antibody [EPR18455], ab188470. Candidate ASOs for promoting DNMT3A expression are presented in Table 2, and include SEQ ID Nos. 121-128. Mutations and haploinsufficiency lead to Tatton-Brown-Rahman syndrome, an overgrowth syndrome characterized by a distinctive facial appearance, tall stature, and intellectual disability. Boosting DNMT3A is specifically contemplated for the treatment or prevention of this syndrome or symptoms thereof or other sequelae of DNMT3A deficiency. Effects of treatment on, for example, cognitive function or development can be determined based on standardized cognitive testing or improvement in other symptoms or parameters as appropriate.

GIGYF2, or GRB10 interacting GYF protein 2, codes for a protein that is involved in the regulation of tyrosine kinase receptor signaling. GIGYF2 sequence is known for a number of species, e.g., human GIGYF2 (NCBI Gene ID: 26058) mRNA (e.g., NM_001103146.3) and polypeptide (e.g., NP_001096616.1), for example for isoform b. Antibodies for specific detection of GIGYF2 are available, for example, from Proteintech—see Anti-GIGYF2 antibody, Cat #24790-1-AP. Candidate ASOs for promoting GIGYF2 expression are presented in Table 2, and include SEQ ID Nos. 129-136. Mutations or haploinsufficiency can lead to complex neurodevelopmental disorder associated with intellectual disability or autism. Boosting GIGYF2 expression is specifically contemplated for the treatment or prevention of such disorder or symptoms thereof, or other sequelae of GIGYF2 deficiency. Effects of treatment on, for example, cognitive function or development can be determined based on standardized cognitive testing or improvement in other symptoms or parameters as appropriate.

LMNA codes for lamin A and lamin C (lamin A/C), which are major protein components of the two-dimensional matrix of the nuclear lamina. Lamins are involved in nuclear stability, chromatin structure, and gene expression. LMNA sequence is known for a number of species, e.g., human LMNA (NCBI Gene ID: 4000) mRNA (e.g., NM_001257374.3) and polypeptide (e.g., NP_001244303.1), for example isoform D. Antibodies for specific detection of LMNA are available, for example, from Abcam—see Anti-Lamin A+Lamin C antibody [EPR4100] nuclear envelope marker, ab108595. Candidate ASOs for promoting LMNA expression are presented in Table 2, and include SEQ ID Nos. 137-144. Mutations or haploinsufficiency can cause dilated cardiomyopathy characterized by ventricular enlargement, reduced systolic function, and arrhythmias. Boosting LMNA expression is specifically contemplated to treat and/or prevent these symptoms or other sequelae of LMNA deficiency, e.g., to restore or maintain sinus rhythm and/or rate control, which are measurable, for example, by electrocardiogram, or evaluation of improvements in other symptoms or parameters as appropriate.

COL1A1 or Collagen Type 1 Alpha 1 chain codes for the pro-alpha1 chains of type 1 collagen, which plays critical roles in most connective tissues including in the bone, cornea, dermis, and tendon. COL1A1 sequence is known for a number of species, e.g., human COL1A1 (NCBI Gene ID: 1277) mRNA (e.g., NM_000088.4) and polypeptide (e.g., NP_000079.2), for example for isoform 1. Antibodies for specific detection of COL1A1 are available, for example, from Novusbio—see Anti-Collagen I antibody NB600-408. Candidate ASOs for promoting COL1A1 expression are presented in Table 2, and include SEQ ID Nos. 145-156. Mutations or haploinsufficiency can lead to osteogenesis imperfecta type 1, which is characterized by low bone mass, bone fragility and susceptibility to fractures after minimal trauma. Boosting COL1A1 is specifically contemplated for the reduction and/or prevention of osteogenesis imperfect type 1 symptoms or other sequelae of COL1A1 deficiency. Symptoms of osteogenesis imperfect type 1 can be measured, for example, by monitoring bone mineral density or evaluation of improvements in other symptoms or parameters as appropriate

FH, encoding fumarate hydratase, is an enzymatic component of the tricarboxylic acid cycle and plays a critical role in energy production. FH sequence is known for a number of species, e.g., human FH (NCBI Gene ID: 2271) mRNA (e.g., NM_000143.4) and polypeptide (e.g., NP_000134.2), for example for isoform 1. Antibodies for specific detection of FH are available, for example, from Abcam—see Anti-FH/Humarase antibody [EPR21104], ab233394. Candidate ASOs for promoting FH expression are presented in Table 2, and include SEQ ID Nos. 157-164. Mutations or haploinsufficiency results in the accumulation of fumaric acid, and can lead to neonatal and early infantile encephalopathy, as well as hereditary leiomyomatosis and renal cancer syndrome (HLRCC), a disorder characterized by predisposition to cutaneous and uterine leiomyomas and early onset renal cancer. Boosting FH expression is specifically contemplated to reduce or limit the symptoms or other sequelae of such deficiency. Efficacy of treatment can be evaluated, for example, by measurement of fumaric acid, evaluation of encephalopathy, and/or evaluation of tumor incidence or growth characteristics or evaluation of improvements in other symptoms or parameters as appropriate.

HMBS codes for an enzyme, Hydroxymethylbilane synthase, that is a member of the hydroxymethylbilane synthase family critical for the heme biosynthetic pathway. HMBS sequence is known for a number of species, e.g., human HMBS (NCBI Gene ID: 3145) mRNA (e.g., NM_000190.4) and polypeptide (e.g., NP_000181.2), for example for isoform 1. Antibodies for specific detection of HMBS are available, for example, from Novusbio—see Anti-HMBS antibody NBP2-33600. Candidate ASOs for promoting HMBS expression are presented in Table 2, and include SEQ ID Nos. 165-172. Mutations or haploinsufficiency can lead to acute intermittent porphyria which is characterized by attacks of gastrointestinal disturbances, abdominal colic, neurological dysfunctions and peripheral neuropathy. Boosting HMBS expression is specifically contemplated for treatment of such acute intermittent porphyria or other sequelae of HMBS deficiency. Efficacy of treatment can be evaluated by measurement of heme levels or biosynthesis, or by monitoring the occurrence of one or more of the symptoms of acute intermittent porphyria or evaluation of improvements in other symptoms or parameters as appropriate.

LEMD3 or Inner nuclear membrane protein MANI/LEM domain containing 3 codes for a LEM domain-containing protein that functions to antagonize transforming growth factor-beta signaling at the inner nuclear membrane. LEMD3 sequence is known for a number of species, e.g., human LEMD3 (NCBI Gene ID: 23592) mRNA (e.g., NM_014319.5) and polypeptide (e.g., NP_055134.2), for example for isoform 1. Antibodies for specific detection of LEMD3 are available, for example, from Novusbio—see Anti-LEMD3 antibody (OTI6C10), NBP2-02211. Candidate ASOs for promoting EHMT1 expression are presented in Table 2, and include SEQ ID Nos. 173-174. Mutations or haploinsufficiency can cause Buschke-Ollendorff syndrome which is a rare disease characterized by osteopoikilosis and skin growths called disseminated connective-tissue nevi. Boosting LEMD3 expression is specifically contemplated to alleviate or prevent the symptoms or development of this syndrome or other sequelae of LEMD3 deficiency. Treatment can be evaluated by the monitoring the incidence of disseminated connective tissue nevi and/or by monitoring bone density, e.g., via bone mineral density tests or evaluation of improvements in other symptoms or parameters as appropriate.

POLD1 codes for the 125 kDa catalytic subunit of DNA polymerase delta (DNA polymerase delta 1) and plays a critical role in polymerase and exonuclease activity during DNA replication and repair. POLD1 sequence is known for a number of species, e.g., human POLD1 (NCBI Gene ID: 5424) mRNA (e.g., NM_001256849.1) and polypeptide (e.g., NP_001243778.1), for example for isoform 1. Antibodies for specific detection of POLD1 are available, for example, from LSBio—see Anti-Human POLD1 Antibody (LS-B1325). Candidate ASOs for promoting POLD1 expression are presented in Table 2, and include SEQ ID Nos. 175-194. Mutations or haploinsufficiency can cause early onset colon cancer. Boosting POLD1 expression is specifically contemplated for the suppression of such early onset colon cancer or other sequellae of POLD1 insufficiency. The incidence of such colon cancer can be evaluated, for example, by colonoscopy or by measurement of cancer-related biomarkers in the stool or evaluation of improvements in other symptoms or parameters as appropriate.

POLE codes for catalytic subunit A of DNA polymerase epsilon, which is critical for repair and chromosomal DNA replication. POLE sequence is known for a number of species, e.g., human POLE (NCBI Gene ID: 5426) mRNA (e.g., NM_006231.4) and polypeptide (e.g., NP_006222.2), for example for isoform 1. Antibodies for specific detection of POLE are available, for example, from Abcam—see Anti-POLE antibody-N-terminal, ab226848 Candidate ASOs for promoting POLE expression are presented in Table 2, and include SEQ ID Nos. 195-202. Mutations or haploinsufficiency can cause colon polyposis. Boosting POLE is specifically contemplated for the treatment or prevention of such colon polyposis or other sequelae of POLE deficiency. The incidence of such colon polyposis can be evaluated, for example, by colonoscopy or by measurement of biomarkers in the stool or evaluation of improvements in other symptoms or parameters as appropriate.

PPP2R5D codes for serine/threonine-protein phosphatase 2A 56 kDa regulatory subunit delta, a regulatory subunit of phosphatase 2A, which is one of the four major Ser/Thr phosphatases and is involved in the control of cell growth and division. PPP2R5D sequence is known for a number of species e.g., human PPP2R5D (NCBI Gene ID: 5528) mRNA (e.g., NM_006245.4) and polypeptide (e.g., NP_006236.1), for example for isoform 1. Antibodies for specific detection of PPP2R5D are available, for example, from LSBio—see Anti-Human PPP2R5D antibody, LS-B9873. Candidate ASOs for promoting PPP2R5D expression are presented in Table 2, and include SEQ ID Nos. 203-204. Mutations and haploinsufficiency can cause neurodevelopmental disorders. Boosting PP2R5D expression is specifically contemplated for the treatment or prevention of symptoms or other sequelae related to such deficiency. Neurodevelopmental disorders and changes in disease can be evaluated, for example, using standardized cognitive testing or other gauges as appropriate.

SF3B4 codes for splicing factor 3b subunit 4, one of the four subunits of the splicing factor 3B, and plays a critical role in prespliceosomal complex function in pre-mRNA splicing. SF3B4 sequence is known for a number of species e.g., human SF3B4 (NCBI Gene ID: 10262) mRNA (e.g., NM_005850.5) and polypeptide (e.g., NP_005841.1), for example for isoform 1. Antibodies for specific detection of SF3B4 are available, for example, from Abcam see Anti-SF3B4 antibody [3A1], ab183483. Candidate ASOs for promoting SF3B4 expression are presented in Table 2, and include SEQ ID Nos. 205-220. Mutations and haploinsufficiency can cause Nager syndrome, a rare condition that is characterized by malformation of the craniofacial skeleton and limbs. Boosting SF3B4 is specifically contemplated to treat, limit or prevent the development of such symptoms or other sequelae of such deficiency, which can be monitored, e.g., via ultrasonography or other imaging.

TDGF1 codes for an epidermal growth factor-related protein, teratocarcinoma-derived growth factor 1, that is an extracellular, membrane-bound signaling protein that plays an essential role in embryonic development and tumor growth. TDGF1 sequence is known for a number of species e.g., human TDGF1 (NCBI Gene ID: 6997) mRNA (e.g., NM_001174136.2) and polypeptide (e.g., NP_001167607.1), for example for isoform 2. Antibodies for specific detection of TDGF1 are available, for example, from Abcam—see Anti-Cripto1/CRIPTO antibody [EPNCIR106A], ab108391. Candidate ASOs for promoting TDGF1 expression are presented in Table 2, and include SEQ ID Nos. 221-222. Mutations and haploinsufficiency can cause neurodevelopmental disorders. Boosting TDGF1 is specifically contemplated to treat, limit or prevent the development of such symptoms or other sequelae of such deficiency. Neurodevelopmental disorders and changes in disease can be evaluated, for example, using standardized cognitive testing or other gauges as appropriate.

ZWILCH codes for an essential component of the mitotic checkpoint, zwilch kinetochore protein, which prevents cells from prematurely exiting mitosis. ZWILCH sequence is known for a number of species e.g., human ZWILCH (NCBI Gene ID: 55055) mRNA (e.g., NM_017975.5) and polypeptide (e.g., NP_060445.3), for example for isoform 1. Antibodies for specific detection of ZWILCH are available, for example, from Abcam see Anti-ZWILCH antibody [EPR18035-28], ab202898. Candidate ASOs for promoting ZWILCH expression are presented in Table 2, and include SEQ ID Nos. 223-230. Mutations and haploinsufficiency can lead to autism, a neurodevelopmental disorder characterized with social interaction and communication. Boosting ZWILCH expression is specifically contemplated for the treatment of such disorder associated with ZWILCH deficiency. Neurodevelopmental symptoms can be evaluated, for example, using standardized neurodevelopmental and cognitive testing.

In certain embodiments, the retained intron locations that are targeted within the sequence of any of the following genes EHMT1, SIRT6, ENG, GCH1, MEN1, SON, TSC2, CDH1, CAMK2B, DNMT3A, GIGYF2, LMNA, COL1A1, FH, HMBS, LEMD3, POLD1, POLE, PPP2R5D, SF3B4, TDGF1, and ZWILCH are listed in Table 1.

One skilled in the art can determine if targeting the intronic region via ASO leads to increased expression e.g., via PCR-based assays or western-blotting to measure the mRNA and protein level, respectively. Enzyme-linked immunosorbent assays (ELISA) or proteomic assays can also be used to assess protein levels. Furthermore, functional assays can be further used to determine if protein expression is increased or if an increase in expression is sufficient to address the effects of the protein deficiency.

### Antisense Oligonucleotides

As discussed herein above, target gene expression can be promoted by administering antisense oligonucleotides complementary to selected region(s) of a retained intron. As used herein, an “antisense oligonucleotide” is a synthetic single-stranded nucleic acid molecule that is complementary to a DNA or RNA sequence, such as that of a retained intron in an RNA transcript. Thus, oligonucleotides are chosen that are sufficiently complementary to the target, i.e., that hybridize sufficiently well and with sufficient specificity in the context of the cellular environment, to give the desired effect. For example, an antisense oligonucleotide can comprise at least 8, at least 10, at least 15, at least 20, at least 25, at least 30, at least 35 or more bases complementary to a portion of the retained intronic region of a particular gene listed in Table 1.

As used herein, the term “complementary” refers to the capacity for specific hydrogen-bonded base pairing between two polynucleotide molecules. It is understood in the art that a complementary nucleotide sequence need not be 100% complementary to that of its target to be specifically hybridizable. An antisense oligonucleotide (ASO) can be at least 80% complementary to (optionally one of at least 85%, 90%, 91%, 92%, 93%, 94%, 95%, 96%, 97%, 98%, 99% or 100% complementary to) the targeted consecutive nucleotides of a target gene intron. In some embodiments, the ASO can contain up to 8 mismatches over a 40 nucleotide molecule, 6 mismatches over a 30 nucleotide molecule, 4 mismatches over a 20 nucleotide molecule, 3 mismatches over a 15 nucleotide molecule, or 2 mismatches over a 10 nucleotide molecule (80% complementarity). In some embodiments, a complementary nucleic acid sequence for purposes of the present disclosure is specifically hybridizable when binding of the sequence to the target molecule (e.g., pre-mRNA) promotes expression of the target protein and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments of any of the aspects, the antisense oligomer consists of from 8 to 40 nucleobases. In some embodiments of any of the aspects, the antisense oligomer consists of from 8 to 40 nucleobases, 8 to 35 nucleobases, 8 to 30 nucleobases, 8 to 25 nucleobases, 8 to 20 nucleobases, 8 to 15 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 40 nucleobases, 12 to 35 nucleobases, 12 to 30 nucleobases, 12 to 25 nucleobases, 12 to 20 nucleobases, or 12 to 15 nucleobases. In some embodiments of any of the aspects, the ASO is at least 80%, at least 85%, at least 90%, at least 95%, at least 98%, at least 99%, or 100%, complementary to the targeted portion of the intron-retaining transcript or mRNA encoding the protein.

In some embodiments of any of the aspects, the antisense oligomer is at least 80%, at least 85%, at least 90%, at least 95%, at least 98%, at least 99%, or 100% identical to a sequence selected from SEQ ID Nos 1-230.

As used herein, the term “nucleotide” refers to an organic molecule that serves as the monomer unit for forming the nucleic acid polymers deoxyribonucleic acid (DNA) and ribonucleic acid (RNA). Conventional, naturally-occurring nucleotides are the building blocks of nucleic acids and are composed of three subunit molecules: a nitrogenous base, a five-carbon sugar, and at least one phosphate group. Nucleotides can be modified. Modifications to nucleotides or oligonucleotides comprised of them can improve, for example, stability, strength of hybridization, and/or cellular delivery or uptake characteristics. Tolerable modifications maintain the ability to hybridize to intron sequence in an RNA transcript and promote expression from a gene that produces transcripts with a retained intron.

The preparation of modified nucleic acids, backbones, and nucleobases is well known in the art. The nucleic acids described herein can be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference, among others. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the T position or 4′ position) or replacement of the sugar, as well as (d) backbone modifications, including modification or replacement of the phosphodiester linkages. Modified nucleotides can include, for example, phosphorothioates, chiral phosphorothioates, phosphorodithioates, phosphotriesters, aminoalkylphosphotriesters, methyl and other alkyl phosphonates including 3 ‘-alkylene phosphonates and chiral phosphonates, phosphinates, phosphoramidates including 3’-amino phosphoramidate and aminoalkylphosphoramidates, thionophosphoramidates, thionoalkylphosphonates, thionoalkylphosphotriesters, and boranophosphates having normal 3′-5′ linkages, 2-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH-CH2-, -CH2-N(CH3)-0-CH2- [known as a methylene (methylimino) or MMI backbone], —CH2-O—N(CH3)-CH2-, -CH2-N(CH3)-N(CH3)-CH2- and —N(CH3)-CH2-CH2- [wherein the native phosphodiester backbone is represented as —O—P—O-CH2-].

In some embodiments of any of the aspects, the ASO comprises an oligonucleotide of 8 to 40 linked nucleotides in length, which oligonucleotide comprises at least one modified nucleotide which comprises a modified sugar moiety. The modification of the sugar moiety can be, for example, a 2′ modification of its sugar moiety. Non-limiting examples include a 2′-O-methyl, 2′-Fluoro, 2′-methoxyethyl, 2′-methoxyethoxy, 2′-dimethylaminooxyethoxy, 2′-dimethylaminoethoxyethoxy, 2′-fluoro or 2′-acetamide modification. Such modification can be on one or more, up to and including on all sugar moieties in the oligonucleotide.

In some embodiments of any of the aspects, the ASO comprises a locked nucleic acid (LNA) nucleobase.

In some embodiments of any of the aspects, the ASO comprises at least one modified backbone linkage. Non-limiting examples of modified linkages include a phosphorothioate linkage or a phosphorodiamidate linkage.

In some embodiments of any of the aspects, the ASO is a morpholino or peptide nucleic acid.

In some embodiments of any of the aspects, the ASO comprises at least one modified base which increases binding affinity for the pre-mRNA target, which increases nuclease resistance of the antisense compound, or which decrease immune-stimulation.

In some embodiments of any of the aspects, the modified base includes methyl-C.

In some embodiments of any of the aspects, the ASO is a mixture of one or more stereopure molecules with a defined sequence.

The activity of a given ASO for promoting intron removal can be assessed, for example, as described in U.S. Pat. No. 9,976,143, or in WO2020/191212, which are incorporated herein by reference. PCR-based assays as described therein can be used to evaluate or demonstrate the activity of a given ASO for promoting splicing to remove a retained intron, and protein-based assays as described therein, such as Western blotting, can be used to evaluate or demonstrate ASO activity in promoting protein expression.

### Formulations

In some embodiments, the antisense oligonucleotides applicable in the compositions and methods described herein are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties, and preparation methods have been described in the published literature. In some embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N—Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In some embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

Pharmaceutical compositions or formulations comprising the antisense oligonucleotide of the described compositions and for use in any of the described methods can be prepared according to conventional techniques well known in the pharmaceutical industry and described in the published literature. In some embodiments, a pharmaceutical composition or formulation for treating a subject comprises an effective amount of any antisense oligomer as described herein, or a pharmaceutically acceptable salt, solvate, hydrate or ester thereof, and a pharmaceutically acceptable diluent. The antisense oligomer of a pharmaceutical formulation can further comprise a pharmaceutically acceptable excipient or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66:1-19 (1977), incorporated herein by reference for this purpose) The salts can be prepared in situ during the final isolation and purification of the compounds, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some embodiments, the compositions are formulated into any of many possible dosage forms including, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension can also contain stabilizers. In some embodiments, a pharmaceutical formulation or composition applicable to the compositions and methods described herein includes, but is not limited to, a solution, emulsion, microemulsion, foam or liposome-containing formulation (e.g., cationic or noncationic liposomes).

The pharmaceutical composition or formulation for use in the methods as described herein can comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the methods and compositions described herein employ a penetration enhancer to effect the efficient delivery of the antisense oligonucleotide, e.g., to aid diffusion across cell membranes and/or enhance the permeability of a lipophilic drug. In some embodiments, the penetration enhancer is a surfactant, fatty acid, bile salt, chelating agent, or non-chelating nonsurfactant.

In some embodiments, the pharmaceutical formulation comprises multiple antisense oligonucleotides. In some embodiments, the antisense oligonucleotide is administered in combination with another drug or therapeutic agent. In some embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” which is incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” which is incorporated herein by reference.

In some embodiments, the antisense oligonucleotides are linked or conjugated with agents that provide desirable pharmaceutical or pharmacodynamic properties. In some embodiments, the antisense oligonucleotide is coupled to a substance, known in the art to promote penetration or transport across the blood-brain barrier, e.g., an antibody to the transferrin receptor. In some embodiments, the antisense oligonucleotide is linked with a viral vector, e.g., to render the antisense compound more effective or increase transport across the blood-brain barrier. In some embodiments, osmotic blood brain barrier disruption is assisted by infusion of sugars, e.g., meso erythritol, xylitol, D (+) galactose, D (+) lactose, D (+) xylose, dulcitol, myo-inositol, L (−) fructose, D (−) mannitol, D (+) glucose, D (+) arabinose, D (−) arabinose, cellobiose, D (+) maltose, D (+) raffinose, L (+) rhamnose, D (+) melibiose, D (−) ribose, adonitol, D (+) arabitol, L (−) arabitol, D (+) fucose, L (−) fucose, D (−) lyxose, L (+) lyxose, and L (−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each of which is incorporated herein by reference.

In some embodiments, two or more ASOs with different chemistries but complementary to the same targeted portion of a retained-intron-containing transcript can be used. In some embodiments, two or more ASOs that are complementary to different targeted portions of the retained-intron-containing transcript can be used.

### Dosage and Administration

### Dosage

The dosage of an agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Typically, the dosage ranges are between 0.001 mg/kg body weight to 5 g/kg body weight, inclusive. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

In some embodiments, the pharmaceutical compositions can conveniently be presented in unit dosage form. Pharmaceutical preparations can be prepared in unit dosage form according to standard procedures of pharmaceutical formulation. The quantity of active compound per unit dose can be varied according to the nature of the active compound and the intended dosage regime. A unit dosage form will typically be adapted to one or more specific routes of administration of the pharmaceutical composition. “Unit dosage form” as the term is used herein refers to a dosage suitable for one administration. By way of example, a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

Liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Suitable diluents and solvents include sterile water, Ringer's solution and isotonic sodium chloride solution, etc. Liquid dosage forms also include solutions or sprays for intranasal administration.

### Administration

In some aspects, the methods described herein for treating a subject comprise administering an ASO. The agents described herein can be administered to a subject in need thereof by any appropriate route which results in an effective treatment in the subject.

The pharmaceutical composition is preferably administered in an amount effective to modulate the expression of the targeted gene by at least 1%, by at least 3%, by at least 5%, by at least 10%, by at least 15%, by at least 20%, by at least 30%, by at least 40%, by at least 50%, by at least 60%, by at least 70%, by at least 80%, by at least 90% or by 100%. It is preferred that the amount administered is an amount effective to maximize the modulation of the expression of the targeted gene while minimizing toxicity.

Administration of an agent described herein (e.g., an ASO) can be performed in a variety of manners, for example, in a single dose, in reoccurring multiple doses, via continuous infusion, via pulsed administration. In one embodiment, an agent described herein can be administered to a subject at least once every 1, 2, 3, 4, 5, 6, 7, 8, 9, 10, 11, 12, 13, 14, 15, 16, 17, 18, 19, 20, 21, 22, 23, or 24 hours; or every 1, 2, 3, 4, 5, 6, or 7 days; or every 1, 2, 3, or 4 weeks; or every 1, 2, 3, 4, 5, 6, 7, 8, 9, 10, 11, or 12 months, or more. It is specifically contemplated herein that the dosing of an agent described herein is determined based on the half-life of the agent, e.g., such that the effect of the agent described herein is maintained at a level that provides continuous, or nearly continuous, effect in the subject.

In one embodiment, the agent described herein is administered at least once. In one embodiment, the agent described herein is administered at least twice. For example, the agent described herein can be administered at least 2, 3, 4, 5, 6, 7, 8, 9, 10, 11, 12, 13, 14, 15, 16, 17, 18, 19, 20, or more times.

In some embodiments, the methods described herein comprise administering an effective amount of the agent to a subject in order to alleviate at least one symptom of a given disease. As used herein, “alleviating at least one symptom of a given disease” refers to ameliorating a condition or symptom associated with that disease. As compared with an equivalent untreated control, such reduction or amelioration is by at least 5%, 10%, 20%, 40%, 50%, 60%, 80%, 90%, 95%, 99% or more as measured by any standard technique. A variety of means for administering an agent as described herein to subjects are known to those of skill in the art. In one embodiment, the agent is administered systemically or locally (e.g., to an affected organ). In one embodiment, the agent is administered intravenously. In one embodiment, the agent is administered continuously, in intervals, or sporadically. The route of administration of the agent will be optimized for the type of agent being delivered (e.g., an ASO), and can be determined by a skilled practitioner.

In one embodiment, the agent is administered continuously (e.g., at constant levels over a period of time). Continuous administration of an agent can be achieved, e.g., by epidermal patches, continuous release formulations, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

Exemplary modes of administration of the ASOs disclosed herein or pharmaceutical compositions comprising them include oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular (including administration to skeletal, diaphragm and/or cardiac muscle), intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the ASOs can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

Aerosol preparations suitable for inhalation can include solutions and solids in powder form, which can be combined with a pharmaceutically acceptable carrier, such as an inert compressed gas. Use of a long-term sustained release implant can be particularly suitable for treatment of chronic conditions such as Alzheimer's disease. Long-term release, as used herein, means that the implant is constructed and arranged to deliver therapeutic levels of the active ingredient for at least 5 days, for at least 10 days, for at least 15 days, for at least 20 days, for at least 30 days, for at least 40 days, for at least 50 days or for at least 60 days. Long-term sustained release implants are known to those of ordinary skill in the art.

Advantageously, the administration of an ASO can be coupled with other treatment methodologies. The ASO-containing agents described herein can also be used in combination in order to achieve the desired therapeutic effect. Certain combinations of agents may act co-operatively, additively or synergistically, when co-administered or when administered sequentially. The antisense treatment can be applied before, after, or in combination with other treatments. As non-limiting examples, for Alzheimer's disease, treatments include but are not limited to cholinesterase inhibitor, Memantine, and/or aducanumab; for Parkinson's disease treatments include but are not limited to Carbidopalevodopa, Dopamine agonists, Catechol O-methyltransferase (COMT) inhibitors, Anticholinergics and/or Amantadine.

In neurodegenerative diseases or disorders, it is envisaged that treatment will be administered continuously over a long period of time to prevent or slow the development of pathology in the brain. Oral dosage forms are particularly preferred for long term therapy and prophylactic treatment because of convenience for the patient, but IV or other parenteral administration can also be used.

In some embodiments, cell-penetration peptides (CPPs) can be used as a transmembrane drug delivery agent for improved delivery of ASOs. CPPs are a class of small cationic peptides of at least 10, or at least 11, or at least 12, or at least 13, or at least 14, or at least 15, or at least 15, or at least 20, or at least 25, or at least 30 amino acids that can be used as transmembrane drug delivery agents through various forms of endocytosis for low-molecular weight compounds, including drugs, imaging agents, oligonucleotides, peptides and proteins. CPPs are also known as ‘protein transduction domains’. CPPs include but are not limited to the peptides Tat or penetratin.

In some embodiments, arginine-rich CPPs can be used for improved delivery of ASOs, e.g. Pep-3, for in vivo delivery.

In some embodiments, RVG-9R, a short peptide derived from rabies virus glycoprotein (RVG) to a nine arginine siRNA packaging peptide (RVG-9R) can be used for delivery of ASOs. RVG-9R permits the transvascular delivery of nucleic acids e.g. ASOs, specifically to target neuronal cells expressing the nicotinic ACh receptor (nAChR) in the brain. As proof of principle, intravenous treatment with RVG-9R-bound siRNA against flavivirus induces robust protection against fatal viral encephalitis in mice, thereby demonstrating systemic delivery of macromolecular oligonucleotides across the blood-brain barrier and specific targeting of the brain of adult mice. This approach is directly applicable to delivery of ASOs as described herein.

Other delivery systems can include time-release, delayed release or sustained release delivery systems. Such systems can avoid or limit the frequency of repeated administrations of the therapeutic agents described herein, increasing convenience to the subject and the physician. Many types of delayed or extended release delivery systems are available and known to those of ordinary skill in the art. They include, for example, polymer based systems such as poly(lactide-glycolide), copolyoxalates, polycaprolactones, polyesteramides, polyorthoesters, polyhydroxybutyric acid, and polyanhydrides. Microcapsules of such polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the therapeutic agent(s) of described herein are contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The most suitable route in any given case will depend on the nature and severity of the condition being treated, ameliorated, and/or prevented and on the nature of the particular ASO or ASO formulation being used. Additionally, ASOs permit one to administer more than one ASO e.g. multiple ASO (e.g. an ASO cocktail).

### Efficacy

The efficacy of a composition in, e.g. the treatment of a condition described herein, or to induce a response as described herein can be determined by the skilled clinician. However, a treatment is considered “effective treatment,” as the term is used herein, if one or more of the signs or symptoms of a condition described herein is altered in a beneficial manner, other clinically accepted symptoms are improved or ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein. When using an experimental animal model, efficacy of treatment is evidenced when a statistically significant change in a marker is observed.

In some embodiments, evaluation of efficacy can comprise monitoring a change in the amount of the fully-spliced target transcript in a sample after administration of an ASO-containing formulation as described herein. In some embodiments, the total amount of the mature mRNA encoding the target protein RNA produced in the cell contacted with the ASO is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, relative to the total amount of the mature mRNA encoding the target protein produced in a control cell.

In some embodiments, the total amount of the target protein produced in the cell or tissue contacted with the ASO is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, relative to the total amount of the target protein produced in a corresponding cell or tissue not contacted with the ASO.

In some embodiments, the ASOs can be used in both veterinary and medical applications.

In vitro and animal model assays permit the assessment of a given dose of a composition. The efficacy of a given dosage combination can also be assessed in an animal model.

The efficacy of an agent described herein, e.g., for the treatment of a disease including, but not limited to Alzheimer's disease, frontotemporal dementia, Kleefstra syndrome, hereditary hemorrhagic telangiectasia, Dopa-responsive dystonia, multiple endocrine neoplasia type 1, ZTTK syndrome, tuberous sclerosis 2, hereditary diffuse gastric adenocarcinoma, an intellectual developmental disorder, Tattan-Brown-Rahman syndrome, dilated cardiomyopathy, osteogenesis imperfecta type 1, hereditary leiomyomatosis renal cancer, acute intermittent porphyria, Buschke-Ollendorff syndrome, early onset colon cancer, colon polyposis, Nager syndrome, or autism, can be determined by the skilled practitioner. However, a treatment is considered “effective treatment,” as the term is used herein, if one or more of the signs or symptoms of a disease or disorder are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., increased target protein levels in the appropriate tissue or cell type. Efficacy can also be measured by a failure of an individual to worsen as assessed by hospitalization, or need for medical interventions (i.e., progression of the symptoms).

Assays well known in the art can be used to test the efficiency of modulation of the target protein expression or activity by the ASO and/or ASOs described herein in both in vitro and in vivo models. The efficiency of modulation of the target's expression or activity in the brain by the ASO and/or ASOs described herein can be assessed by one skilled in the art by measuring mRNA and protein levels of the targeted gene (e.g., using reverse transcription PCR, western blot analysis, and/or enzyme-linked immunosorbent assay (ELISA)).

Efficacy can be assessed in animal models of a condition described herein, for example, a mouse model or an appropriate animal model of a given disease or disorder, as the case may be. When using an experimental animal model, efficacy of treatment is evidenced when a statistically significant change in a marker is observed, e.g. increased target protein levels.

It is contemplated herein that the effects of modulation by the ASO and/or ASOs in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

### EXAMPLES

### Example 1: Identification of ASO Target Genes

It was reasoned that a candidate gene for increasing expression with ASOs should meet the following criteria:
1. A fraction of transcripts retain exactly 1 intron relative to the canonical protein-coding transcript model. This fraction is referred to herein as the fraction retained (FR);
2. Haploinsufficiency of the gene is associated with disease.
   To more precisely articulate which genes satisfy the first criterion that a fraction of transcripts retain exactly one intron relative to the canonical protein-coding transcript model, two publicly-available datasets from samples listed below were downloaded and analyzed:
A. Alzheimer's disease brain (AD)-generated by Pacific Biosciences, Menlo Park, California; additional information regarding the sequencing and analysis is available on the world wide web at //downloads.pacbcloud.com/public/dataset/Alzheimer2019_IsoSeq/; and
B. Universal human reference RNA (UHRR), from a mixture of tissues—also generated by Pacific Biosciences; additional information regarding the sequencing and analysis is available on the world wide web at //downloads.pacbcloud.com/public/dataset/UHR_IsoSeq/.

Iso-Seq reads are advantageous because they span the whole length of the mRNA transcript, permitting direct identification of IR and more specifically the case described in criterion (1)—i.e., a fraction of transcripts retain exactly one intron relative to the canonical protein-coding transcript model. Using this relatively small amount of long read data rather than short-read data was crucial, because it permitted one to directly observe the presence of exactly one intron retention (IR) event in transcripts that otherwise matched the canonical protein coding sequence. Over short-read data, this would not have been possible, because without long range information from the same transcript one could not have verified that separate IR events were happening on different transcripts, or that putative IR events were actually occurring in transcripts that otherwise matched the protein coding sequence. Both of these were important filtering criteria for identifying candidates because an ASO targeted to a single IR event in a transcript that retains several will likely fail, as will an ASO targeted to a noncoding transcript.

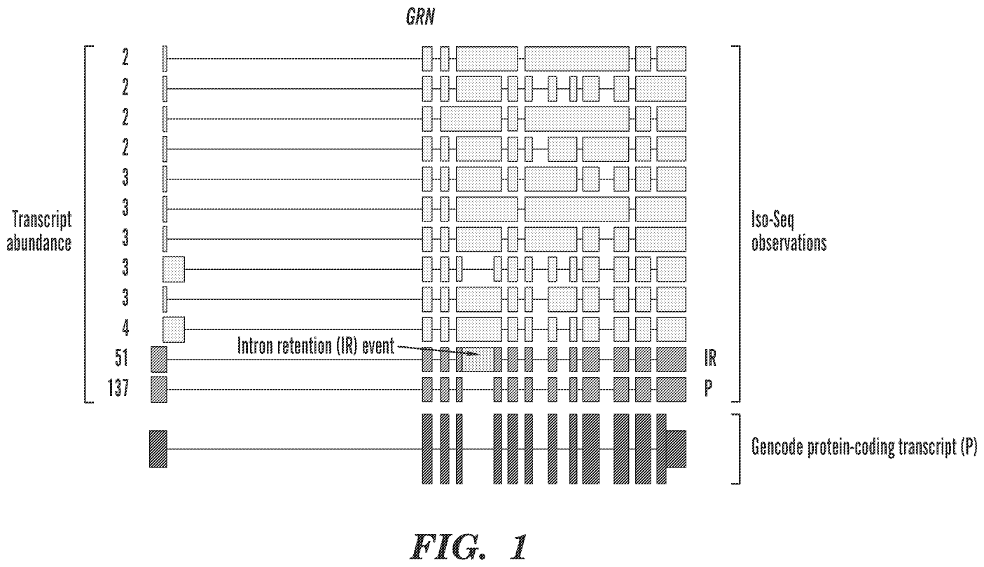

The analysis of the Iso-Seq data yielded a large set of genes meeting the criteria above, and, encouragingly, re-identified GRN (FIG. 1). Table 1 provides a set of hits satisfying the first criterion that also satisfy the second criterion of haploinsufficiency being related to disease. Some of the identified genes have relatively low FR (<0.01). However, for progranulin, the increase in expression conferred by ASOs exceeded what would have been expected from abrogation of intron retention alone (see, e.g., WO2020/191212); thus, ASO treatment of low-FR cases can still provide considerable therapeutic benefit.

Through use of the Alzheimer's disease brain gene expression database, the approach set out here focused on genes more likely to be involved in diseases of the nervous system. However, genes in which expression in the brain involves a retained intron may well involve the retention of an intron in other tissues, and such retention can be associated with diseases affecting those other tissues. Thus, the identified genes and their retained introns represent targets for ASO-mediated upregulation and treatment for a disease or disorder associated with insufficient expression of those genes wherever their expression is affected by intron retention. That is, intron retention identified in the Alzheimer's disease database may be present and targetable in other tissues. It is also noted that the causative involvement of insufficient expression of certain genes with particular diseases may be strengthened when additional data are evaluated, or, for that matter, that genes characterized by or involving intron retention in one or more cell types or tissues can be implicated in completely different diseases.

### Example 2: ASO Design

Within the introns identified as described above, the methodology previously applied to the development of GRN-targeting ASOs was used to identify sequences within the retained introns that are most likely to permit ASO-mediated up-regulation. The intron sequences were evaluated using an approach adapted from Rosenberg et al., Cell 163:698-711 (2015) to identify sequences likely to be involved in suppression of splicing or reduced splicing efficiency. Rosenberg et. al. calculated a score describing the tendency of each hexamer motif to cause retention or splicing-out of minigene introns. This model was applied to intronic sequences in the identified genes showing intron retention (Table 1). Thus, hexamer motifs tiled across each whole retained intron were evaluated for intronic splice suppression scores, and regions of the retained intron in which hexamer motifs have the highest average score were identified as candidate targets for ASOs. These sequences were tiled with, for example, 19-mer ASOs, which can then be tested to demonstrate activity in promoting target protein expression, e.g., in a suitable in vitro cell culture model, and/or in an in vivo animal model. Table 2 provides candidate ASOs identified in this manner for each gene target listed in Table 1.

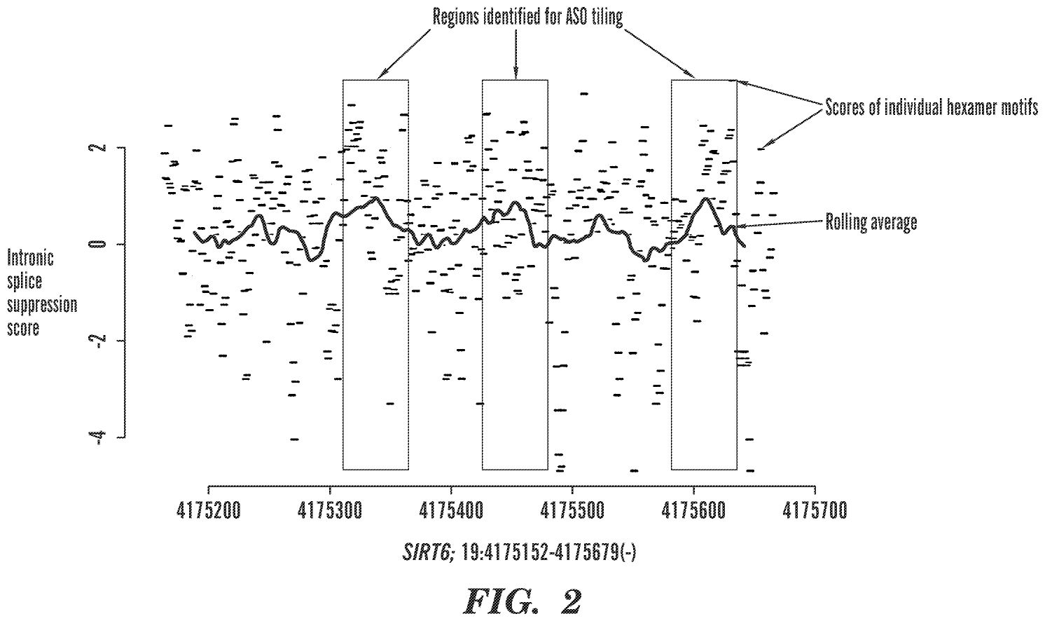

While the intronic subsequences identified herein (light green in FIG. 2) provide the best candidates based on the approach described, it is possible that other intronic subsequences can also provide increased target gene expression. Thus, ASOs targeted to intronic sequence outside of these identified subsequences may also boost protein expression when targeted with ASOs.

TABLE 1

| Gene | HS | Disease | Data | Obs. | FR | Intron location |
|---|---|---|---|---|---|---|
| EHMT1 | 3 | Kleefstra syndrome | AD | 73 ( [symbol] 3) | 0.3 [symbol] (0.31) | 9:137814509-137815946(+) |
| SIRT6 | *** | Aging, Alzheimer's | AD | 1 [symbol] | 0.44 | 19:4175152-4175679(−) |
| | | disease, others [symbol] | | | | |
| ENG* | 3 | Hereditary Hemorrhagic | AD | 1 [symbol] | 0.02 | 9:127816054-127817148(−) |
| | | Telangiectasia | | | 0.01 | 9:127818833-127819621(−) |
| GCH1 | 3 | Dopa-responsive | AD | 1 [symbol] | 0.13 | 14:54845853-54847098(−) |
| | | dystonia | | | | |
| MEN1 | 3 | Multiple endocrine | AD | 47 (1 [symbol] 3) | 0.17 (0.0 [symbol] ) | 11:64805771-64806231(−) |
| | | neoplasia type 1 | | | | |
| SON | 3 | ZTTK syndrome | AD | 10 (14) | 0. [symbol] 0 (0.3 [symbol] ) | 21:33559776-33559876(+) |
| TSC2** | 3 | Tuberous sclerosis 2 | AD | 17 (7 [symbol] ) | 0.12 (0.0 [symbol] ) | 16:2053453-2054295(+) |
| | | | | [symbol] (7 [symbol] ) | 0.03 (0.0 [symbol] ) | |
| CDH1 | 3 | Hereditary diffuse | AD | 11 | 0.3 [symbol] | 16:68811860-68812134(+) |
| | | gastric adenocarcinoma | | | | |
| CAMK2B* | 2 | Mental retardation | AD | 4 [symbol] 7 ( [symbol] 04) | 0.01 (<0.01) | 7:44220711-44220825(−) |
| | | | | | 0.01 (<0.01) | 7:44241784-44242217(−) |
| DNMT3A | 1 | Tattan-Brown-Rahman | AD | 1 [symbol] (22) | 0.11 (0.0 [symbol] ) | 2:25247750-25248036(−) |
| | | syndrom | | | | |
| GIGYF2 | 2 | Complex | AD | 8 (110) | 0.2 [symbol] (0.02) | 2:232809812-232811243(+) |
| | | neurodevelopmental | | | | |
| | | disorder | | | | |
| LMNA | 2 | Dilated cardiomyopathy | AD | [symbol] 3 (2 [symbol] ) | 0.13 (0.03) | 1:156138758-156139079(+) |
| COL1A1* | 3 | Osteogenesis imperfecta | UHRR | 13 [symbol] 0 | 0.02 | 17:50194042-50194129(−) |
| | | type 1 | | | 0.01 | 17:50186923-50187014(−) |
| | | | | | <0.01 | 17:50194829-50195046(−) |
| FH | 3 | Hereditary | UHRR | [symbol] 32 | 0.01 | 1:241508786-241511966(−) |
| | | lelomyomatosis | | | | |
| | | and renal cell cancer | | | | |
| HMBS | 3 | Acute intermittent | UHRR | 3 [symbol] (44 [symbol] ) | 0.04 (0.03) | 11:119092164-119092403(+) |
| | | porphyria | | | | |
| LEMD3 | 3 | Buschke-ollendorff | UHRR | 10 [symbol] | 0.02 | 12:65245775-65245860(+) |
| | | syndrome | | | | |
| POLD1* | 1 | Early onset colon | UHRR | [symbol] 1 ( [symbol] 4 [symbol] ) | 0.01 (<0 01) | 19:50415827-50416395(+) |
| | | cancer, colon polyposis | | | <0.01 (<0.01) | 19:50416529-50416609(+) |
| POLE | 1 | Early onset colon | UHRR | 2 [symbol] (30) | 0.12 (0.10) | 12:132661165-132661526(−) |
| | | cancer, colon polyposis | | | | |
| PPP2R5D | 1 | Neurodevelopmental | UHRR | 22 [symbol] ( [symbol] 77) | 0.02 (0.01) | 6:43008066-43008200(+) |
| | | disorders | | | | |
| SF3B4* | 3 | Nager syndrome | UHRR | 104 [symbol] (104 [symbol] ) | 0.01 (0.01) | 1:149924015-149925835(−) |
| | | | | | <0.01 (<0.01) | 1:149926043-149926375(−) |
| TDGF1 | 1 | Neurodevelopmental | UHRR | 1 [symbol] (207) | 0.02 (0.01) | 3:46579854-46579950(+) |
| | | disorders | | | | |
| ZWILCH | 1 | Autism | UHRR | 47 [symbol] ( [symbol] 3) | 0.02 (0.01) | 15:66527384-66527856(+) |

[symbol]

indicates data missing or illegible when filed

TABLE 2

| Gene | Intron Location | ASOs |
|---|---|---|
| EHMT1 | 9:137814509- | SEQ ID NO: 1 TTCAGCTGGTTGGGGCCTC; |
| | 137815946(+) | SEQ ID NO: 2 GGTTGCTCTGCTTTCTTCA; |
| | | SEQ ID NO: 3 GGCTGCCAGCTCCTGGGTT; |
| | | SEQ ID NO: 4 GACGCCTGCACTGTTTTTG; |
| | | SEQ ID NO: 5 AGCGCTGGGGTCACAGACG; |
| | | SEQ ID NO: 6 CGACGCTGCCACGACAGCG; |
| | | SEQ ID NO: 7 CTTTCTTCAGCTGGTTGGG; |
| | | SEQ ID NO: 8 TCCTGGGTTGCTCTGCTTT; |
| | | SEQ ID NO: 9 ACCGAGGCTGCCAGCTCCT; |
| | | SEQ ID NO: 10 TCACAGACGCCTGCACTGT; |
| | | SEQ ID NO: 11 ACGACAGCGCTGGGGTCAC; |
| | | SEQ ID NO: 12 CTCTGCTTTCTTCAGCTGG; |
| | | SEQ ID NO: 13 CCAGCTCCTGGGTTGCTCT; |
| | | SEQ ID NO: 14 CTGCACTGTTTTTGCAGAC; |
| | | SEQ ID NO: 15 TGGGGTCACAGACGCCTGC; |
| | | SEQ ID NO: 16 CTGCCACGACAGCGCTGGG |
| SIRT6 | 19:4175152- | SEQ ID NO: 17 TCTGCGGTCCGTTGGCGGG; |
| | 4175679(−) | SEQ ID NO: 18 CGGGGTCATTGATCTGGGC; |
| | | SEQ ID NO: 19 GGGCGGGCCCTTAGACACT; |
| | | SEQ ID NO: 20 GCCTCAGCCACTGTCCCAT; |
| | | SEQ ID NO: 21 CCATGCTGGAGCTGGCTGT; |
| | | SEQ ID NO: 22 CTGTGTTTCTGCTGTCGTC; |
| | | SEQ ID NO: 23 AGCACTGGATAGAGGAGAC; |
| | | SEQ ID NO: 24 AGACACCTGGCGGAAGCCT; |
| | | SEQ ID NO: 25 GGTCCGTTGGCGGGGTCAT; |
| | | SEQ ID NO: 26 TCATTGATCTGGGCGGGCC; |
| | | SEQ ID NO: 27 GGCCCTTAGACACTTCCGG; |
| | | SEQ ID NO: 28 AGCCACTGTCCCATGCTGG; |
| | | SEQ ID NO: 29 CTGGAGCTGGCTGTGTTTC; |
| | | SEQ ID NO: 30 CCGGCTGGGCAGCACTGGA; |
| | | SEQ ID NO: 31 TGGATAGAGGAGACACCTG; |
| | | SEQ ID NO: 32 CCTGGCGGAAGCCTTGGTT; |
| | | SEQ ID NO: 33 GTTGGCGGGGTCATTGATC; |
| | | SEQ ID NO: 34 GATCTGGGCGGGCCCTTAG; |
| | | SEQ ID NO: 35 TGTGAGCCTCAGCCACTGT; |
| | | SEQ ID NO: 36 CTGTCCCATGCTGGAGCTG; |
| | | SEQ ID NO: 37 GCTGGCTGTGTTTCTGCTG; |
| | | SEQ ID NO: 38 TGGGCAGCACTGGATAGAG; |
| | | SEQ ID NO: 39 AGAGGAGACACCTGGCGGA; |
| | | SEQ ID NO: 40 CGGAAGCCTTGGTTTCCTT |
| ENG | 9:127816054- | SEQ ID NO: 41 CTTCTTGGTCCTGGGCTGG; |
| | 127817148(−) | SEQ ID NO: 42 CTGGGAGTGGAGGCTCCAT; |
| | | SEQ ID NO: 43 CCATCCTGGCCTGGATGGG; |
| | | SEQ ID NO: 44 TGGTCCTGGGCTGGGAGTG; |
| | | SEQ ID NO: 45 AGTGGAGGCTCCATCCTGG; |
| | | SEQ ID NO: 46 CTGGCCTGGATGGGGTGGC; |
| | | SEQ ID NO: 47 CTGGGCTGGGAGTGGAGGC; |
| | | SEQ ID NO: 48 AGGCTCCATCCTGGCCTGG |
| | 9:127818833- | SEQ ID NO: 49 GAGACGGAGTCTCGCTCTG; |
| | 127819621(−) | SEQ ID NO: 50 TCTGTCGCCCAGGCTGGAG; |
| | | SEQ ID NO: 51 GGAGTGCAGTGGTGCGATC; |
| | | SEQ ID NO: 52 GGAGTCTCGCTCTGTCGCC; |
| | | SEQ ID NO: 53 CGCCCAGGCTGGAGTGCAG; |
| | | SEQ ID NO: 54 GCAGTGGTGCGATCTCAGC; |
| | | SEQ ID NO: 55 CTCGCTCTGTCGCCCAGGC; |
| | | SEQ ID NO: 56 AGGCTGGAGTGCAGTGGTG |
| GCH1 | 14:54845853- | SEQ ID NO: 57 TGGAAGGCTGAGGCTGCAG; |
| | 54847098(−) | SEQ ID NO: 58 GCAGTGAGCCGAGATTGCA; |
| | | SEQ ID NO: 59 TGCACCACTGCACTCCAGC; |
| | | SEQ ID NO: 60 GGCTGAGGCTGCAGTGAGC; |
| | | SEQ ID NO: 61 GAGCCGAGATTGCACCACT; |
| | | SEQ ID NO: 62 CACTGCACTCCAGCCTGGG; |
| | | SEQ ID NO: 63 AGGCTGCAGTGAGCCGAGA; |
| | | SEQ ID NO: 64 GAGATTGCACCACTGCACT |
| MEN1 | 11:64805771- | SEQ ID NO: 65 GGGCGTGGGAGCCAGGCCT; |
| | 64806231(−) | SEQ ID NO: 66 GCCTCAGTCCTGGACGAGG; |
| | | SEQ ID NO: 67 GAGGGTGGTTGGAAACTGA; |
| | | SEQ ID NO: 68 TGGGAGCCAGGCCTCAGTC; |
| | | SEQ ID NO: 69 AGTCCTGGACGAGGGTGGT; |
| | | SEQ ID NO: 70 TGGTTGGAAACTGATGGAG; |
| | | SEQ ID NO: 71 GCCAGGCCTCAGTCCTGGA; |
| | | SEQ ID NO: 72 TGGACGAGGGTGGTTGGAA |
| SON | 21:33559776- | SEQ ID NO: 73 AATGGCAGATTCAGGTATA; |
| | 33559876(+) | SEQ ID NO: 74 AATGAAATGGCAGATTCAG |
| TSC2 | 16:2053453- | SEQ ID NO: 75 AGGGGATGACACAGCTCCT; |
| | 2054295(+) | SEQ ID NO: 76 CGGGTGGCGCAGAGCAGGG; |
| | | SEQ ID NO: 77 CGGGACCTGGCACAGCGGG; |
| | | SEQ ID NO: 78 GCAACTGCTGTGTGTCGGG; |
| | | SEQ ID NO: 79 AGCGGCGTATGGGGAGCAA; |
| | | SEQ ID NO: 80 ACCGGCCGACCGCAGAGCG; |
| | | SEQ ID NO: 81 AGAGCAGGGGATGACACAG; |
| | | SEQ ID NO: 82 CACAGCGGGTGGCGCAGAG; |
| | | SEQ ID NO: 83 TGTGTCGGGACCTGGCACA; |
| | | SEQ ID NO: 84 GGGGAGCAACTGCTGTGTG; |
| | | SEQ ID NO: 85 CGCAGAGCGGCGTATGGGG; |
| | | SEQ ID NO: 86 GATGCACCGGCCGACCGCA; |
| | | SEQ ID NO: 87 GGCGCAGAGCAGGGGATGA; |
| | | SEQ ID NO: 88 CCTGGCACAGCGGGTGGCG; |
| | | SEQ ID NO: 89 TGCTGTGTGTCGGGACCTG; |
| | | SEQ ID NO: 90 CGTATGGGGAGCAACTGCT; |
| | | SEQ ID NO: 91 CCGACCGCAGAGCGGCGTA; |
| | | SEQ ID NO: 92 GGCCGGATGCACCGGCCGA |
| CDH1 | 16:68811860- | SEQ ID NO: 93 ACGGAACCTCTGGGCACAA; |
| | 68812134(+) | SEQ ID NO: 94 GCCTGTCTTCTAGGCACGG; |
| | | SEQ ID NO: 95 GCTTCATGCCAGCCTGCCT; |
| | | SEQ ID NO: 96 TAGGCACGGAACCTCTGGG; |
| | | SEQ ID NO: 97 AGCCTGCCTGTCTTCTAGG; |
| | | SEQ ID NO: 98 CATGTGCTTCATGCCAGCC; |
| | | SEQ ID NO: 99 TCTTCTAGGCACGGAACCT; |
| | | SEQ ID NO: 100 ATGCCAGCCTGCCTGTCTT |
| CAMK2B | 7:44220711- | SEQ ID NO: 101 CCCACATCCTTGTCCTGCA; |
| | 44220825(−) | SEQ ID NO: 102 GTGAGGAGGGGCCCCGTGG; |
| | | SEQ ID NO: 103 ATCCTTGTCCTGCACAGCC; |
| | | SEQ ID NO: 104 TCCAAGTGAGGAGGGGCCC; |
| | 7:44241784- | SEQ ID NO: 105 TGGCAGCCGAGCCCGAGGC; |
| | 44242217(−) | SEQ ID NO: 106 AGGCACAGGGGAGAGGCCA; |
| | | SEQ ID NO: 107 GCCAGGGTGGCAAGGCTTG; |
| | | SEQ ID NO: 108 TTGGGGTCCTTGCCAGGGG; |
| | | SEQ ID NO: 109 GGGGAGTGGTCTGAGGCCA; |
| | | SEQ ID NO: 110 GCCATCTCCACCAGGAGCC; |
| | | SEQ ID NO: 111 GCCGAGCCCGAGGCACAGG; |
| | | SEQ ID NO: 112 CAGGGGAGAGGCCAGGGTG; |
| | | SEQ ID NO: 113 GGTGGCAAGGCTTGTGGCA; |
| | | SEQ ID NO: 114 GTCCTTGCCAGGGGAGTGG; |
| | | SEQ ID NO: 115 GTGGTCTGAGGCCATCTCC; |
| | | SEQ ID NO: 116 GCCCGAGGCACAGGGGAGA; |
| | | SEQ ID NO: 117 GAGAGGCCAGGGTGGCAAG; |
| | | SEQ ID NO: 118 CCCTCTTGGGGTCCTTGCC; |
| | | SEQ ID NO: 119 TGCCAGGGGAGTGGTCTGA; |
| | | SEQ ID NO: 120 CTGAGGCCATCTCCACCAG |
| DNMT3A | 2:25247750- | SEQ ID NO: 121 CGAGCGGCCCGTGGGAGAT; |
| | 25248036(−) | SEQ ID NO: 122 AGATGGAGAGAGGAGAGCA; |
| | | SEQ ID NO: 123 AGCAGGACGGGAGGAGCTG; |
| | | SEQ ID NO: 124 GGCCCGTGGGAGATGGAGA; |
| | | SEQ ID NO: 125 GAGAGAGGAGAGCAGGACG; |
| | | SEQ ID NO: 126 GACGGGAGGAGCTGGCAGT; |
| | | SEQ ID NO: 127 GTGGGAGATGGAGAGAGGA; |
| | | SEQ ID NO: 128 AGGAGAGCAGGACGGGAGG |
| GIGYF2 | 2:232809812- | SEQ ID NO: 129 TTCGAGGCCAGCCTCGCCA; |
| | 232811243(+) | SEQ ID NO: 130 ACTTGAGGCTGGGAGTTCG; |
| | | SEQ ID NO: 131 CGAGGCAGGCAGATCACTT; |
| | | SEQ ID NO: 132 GGGAGTTCGAGGCCAGCCT; |
| | | SEQ ID NO: 133 AGATCACTTGAGGCTGGGA; |
| | | SEQ ID NO: 134 GGAGGCGAGGCAGGCAGAT; |
| | | SEQ ID NO: 135 AGGCTGGGAGTTCGAGGCC; |
| | | SEQ ID NO: 136 CAGGCAGATCACTTGAGGC |
| LMNA | 1:156138758- | SEQ ID NO: 137 GCTGTGGGGAGGAGGCTCC; |
| | 156139079(+) | SEQ ID NO: 138 CTGTCTAGGACTCAGGCTG; |
| | | SEQ ID NO: 139 GGATGCAGAGGTGGGCTGT; |
| | | SEQ ID NO: 140 CTCAGGCTGTGGGGAGGAG; |
| | | SEQ ID NO: 141 GTGGGCTGTCTAGGACTCA; |
| | | SEQ ID NO: 142 GGGCAGGATGCAGAGGTGG; |
| | | SEQ ID NO: 143 TAGGACTCAGGCTGTGGGG; |
| | | SEQ ID NO: 144 CAGAGGTGGGCTGTCTAGG |
| COL1A1 | 17:50194042- | SEQ ID NO: 145 GCAGCAGTGAGGACAGCAG; |
| | 50194129(−) | SEQ ID NO: 146 AGTGAGGACAGCAGGGAGG |
| | 17:50186923- | SEQ ID NO: 147 GGGGCTGGAGGGCCATGAG; |
| | 50187014(−) | SEQ ID NO: 148 TGGAGGGCCATGAGCAGAG |
| | 17:50194829- | SEQ ID NO: 149 AGAGCCGGCTGAGGCTGGG; |
| | 50195046(−) | SEQ ID NO: 150 TGGGCCTCCAGTGTCAGGG; |
| | | SEQ ID NO: 151 AGGGGTTCCTGGGGGTGTG; |
| | | SEQ ID NO: 152 CGGCTGAGGCTGGGCCTCC; |
| | | SEQ ID NO: 153 CTCCAGTGTCAGGGGTTCC; |
| | | SEQ ID NO: 154 TTCCTGGGGGTGTGGCAGG; |
| | | SEQ ID NO: 155 GAGGCTGGGCCTCCAGTGT; |
| | | SEQ ID NO: 156 GTGTCAGGGGTTCCTGGGG |
| FH | 1:241508786- | SEQ ID NO: 157 CCTCAGCACTTTCAGAGGC; |
| | 241511966(−) | SEQ ID NO: 158 AGGCTGAGGCAGGAGGATC; |
| | | SEQ ID NO: 159 GATCAATTGAGGCCAGGAG; |
| | | SEQ ID NO: 160 GCACTTTCAGAGGCTGAGG; |
| | | SEQ ID NO: 161 GAGGCAGGAGGATCAATTG; |
| | | SEQ ID NO: 162 ATTGAGGCCAGGAGTTTAA; |
| | | SEQ ID NO: 163 TTCAGAGGCTGAGGCAGGA; |
| | | SEQ ID NO: 164 AGGAGGATCAATTGAGGCC |
| HMBS | 11:119092164- | SEQ ID NO: 165 AAGCTTCTTGGTTGAGAAC; |
| | 119092403(+) | SEQ ID NO: 166 AGAAGGTTGTGAGACAAGC; |
| | | SEQ ID NO: 167 TGGGGAAGCAGATGCAGAA; |
| | | SEQ ID NO: 168 GAGACAAGCTTCTTGGTTG; |
| | | SEQ ID NO: 169 GATGCAGAAGGTTGTGAGA; |
| | | SEQ ID NO: 170 TATTCTGGGGAAGCAGATG; |
| | | SEQ ID NO: 171 GTTGTGAGACAAGCTTCTT; |
| | | SEQ ID NO: 172 AAGCAGATGCAGAAGGTTG |
| LEMD3 | 12:65245775- | SEQ ID NO: 173 CAAAACATGAAATTCAATA; |
| | 65245860(+) | SEQ ID NO: 174 AAATCCAAAACATGAAATT |
| POLD1 | 19:50415827- | SEQ ID NO: 175 TCAGGTCACCACGGAAGGG; |
| | 50416395(+) | SEQ ID NO: 176 GAGTCTGTAGGGCTCTCAG; |
| | | SEQ ID NO: 177 AGTCTCTGGGCCACAGAGT; |
| | | SEQ ID NO: 178 GGTCGGAGGTCACGGAGTC; |
| | | SEQ ID NO: 179 CTGGGTCTGGGATGGGGTC; |
| | | SEQ ID NO: 180 TGACATGGGGGGGGCCTGG; |
| | | SEQ ID NO: 181 GGCTCTCAGGTCACCACGG; |
| | | SEQ ID NO: 182 CCACAGAGTCTGTAGGGCT; |
| | | SEQ ID NO: 183 CACGGAGTCTCTGGGCCAC; |
| | | SEQ ID NO: 184 GATGGGGTCGGAGGTCACG; |
| | | SEQ ID NO: 185 GGGGCCTGGGTCTGGGATG; |
| | | SEQ ID NO: 186 GGCTGTGACATGGGGGGGG; |
| | | SEQ ID NO: 187 TGTAGGGCTCTCAGGTCAC; |
| | | SEQ ID NO: 188 CTGGGCCACAGAGTCTGTA; |
| | | SEQ ID NO: 189 GAGGTCACGGAGTCTCTGG; |
| | | SEQ ID NO: 190 TCTGGGATGGGGTCGGAGG; |
| | | SEQ ID NO: 191 TGGGGGGGGCCTGGGTCTG; |
| | | SEQ ID NO: 192 CTGCGGGCTGTGACATGGG |
| | 19:50416529- | SEQ ID NO: 193 AGGTGGTGGGCCGGTGATC; |
| | 50416609(+) | SEQ ID NO: 194 GAGGCAGGTGGTGGGCCGG |
| POLE | 12:132661165- | SEQ ID NO: 195 CACAGCAGTGGCAAGGAGC; |
| | 132661526(−) | SEQ ID NO: 196 GAGCGCTGGGGAGCCACCA; |
| | | SEQ ID NO: 197 ACCAGCTGTGCCTCATCCT; |
| | | SEQ ID NO: 198 CAGTGGCAAGGAGCGCTGG; |
| | | SEQ ID NO: 199 CTGGGGAGCCACCAGCTGT; |
| | | SEQ ID NO: 200 CTGTGCCTCATCCTCTCTG; |
| | | SEQ ID NO: 201 GCAAGGAGCGCTGGGGAGC; |
| | | SEQ ID NO: 202 GAGCCACCAGCTGTGCCTC; |
| PPP2R5D | 6:43008066- | SEQ ID NO: 203 AGCAGAAGGTTTTGCTCCT; |
| | 43008200(+) | SEQ ID NO: 204 TCAGTAGCAGAAGGTTTTG |
| SF3B4 | 1:149924015- | SEQ ID NO: 205 ACTCAGGAGGCTGAGGTGG; |
| | 149925835(−) | SEQ ID NO: 206 GTGGGAGGATCACTTGGGC; |
| | | SEQ ID NO: 207 GGGCCTGGGACGTCAAGGC; |
| | | SEQ ID NO: 208 GGAGGCTGAGGTGGGAGGA; |
| | | SEQ ID NO: 209 AGGATCACTTGGGCCTGGG; |
| | | SEQ ID NO: 210 TGGGACGTCAAGGCTACAG; |
| | | SEQ ID NO: 211 CTGAGGTGGGAGGATCACT; |
| | | SEQ ID NO: 212 CACTTGGGCCTGGGACGTC |
| | 1:149926043- | SEQ ID NO: 213 ATGTCTTTAAAGAGCCTGT; |
| | 149926375(−) | SEQ ID NO: 214 CTGTCCTGATCTGGCCTCT; |
| | | SEQ ID NO: 215 CTCTCCAGGCAGGGTGAGC; |
| | | SEQ ID NO: 216 TTTAAAGAGCCTGTCCTGA; |
| | | SEQ ID NO: 217 CTGATCTGGCCTCTCCAGG; |
| | | SEQ ID NO: 218 CAGGCAGGGTGAGCTCTTT; |
| | | SEQ ID NO: 219 AGAGCCTGTCCTGATCTGG; |
| | | SEQ ID NO: 220 CTGGCCTCTCCAGGCAGGG; |
| TDGF1 | 3:46579854- | SEQ ID NO: 221 CTCTCCTCTCCACGGCTCC; |
| | 46579950(+) | SEQ ID NO: 222 TTTCTCTCTCCTCTCCACG |
| ZWILCH | 15:66527384- | SEQ ID NO: 223 TGTGAGGATGAACGGAGGT; |
| | 66527856(+) | SEQ ID NO: 224 TCCATCAGGTGGGGGTGTG; |
| | | SEQ ID NO: 225 AGATTATAATAGATATCCA; |
| | | SEQ ID NO: 226 GGGGGTGTGAGGATGAACG; |
| | | SEQ ID NO: 227 AGATATCCATCAGGTGGGG; |
| | | SEQ ID NO: 228 GGTGGAGATTATAATAGAT; |
| | | SEQ ID NO: 229 CAGGTGGGGGTGTGAGGAT; |
| | | SEQ ID NO: 230 ATAATAGATATCCATCAGG |

1. A method of increasing, in a cell, the expression of a polypeptide from a gene selected from those genes listed in Table 1, the method comprising introducing to the cell an oligonucleotide comprising antisense sequence complementary to a retained intron of a transcript produced by the gene, wherein the oligonucleotide increases expression of the polypeptide in the cell.

2. (canceled)

3. The method of claim 1, wherein the antisense sequence complementary to a retained intron is at least 8 nucleotides in length.

4. (canceled)

5. (canceled)

6. The method of claim 1, wherein the oligonucleotide comprises one or more of:
a. at least one modified nucleotide which comprises a modified sugar moiety;
b. a 2′ modification of a sugar moiety;
c. a 2′-O-methyl, 2′-methoxyethoxy, 2′-O-methoxyethyl, 2′-dimethylaminooxyethoxy, 2′-dimethylaminoethoxyethoxy, 2′-fluoro, or 2′-acetamide modification on every sugar moiety;
d. a locked nucleic acid (LNA) nucleobase; and
e. at least one modified linkage.

7-72. (canceled)

73. A composition comprising an oligonucleotide of any one of SEQ ID NOS 1-230 or a sequence at least 80% identical thereto, wherein the oligonucleotide comprises at least one modified sugar moiety or modified linkage.

74. The composition of claim 73, wherein the oligonucleotide comprises one or more of:
a. at least one modified nucleotide which comprises a modified sugar moiety;
b. a 2′ modification of a sugar moiety;
c. a 2′-O-methyl, 2′-methoxyethoxy, 2′-O-methoxyethyl, 2′-dimethylaminooxyethoxy, 2′-dimethylaminoethoxyethoxy, 2′-fluoro, or 2′-acetamide modification on every sugar moiety;
d. a locked nucleic acid (LNA) nucleobase; and
e. at least one modified linkage.

75. A pharmaceutical composition comprising a composition of claim 73 and a pharmaceutically acceptable carrier.

76-97. (canceled)

98. The method of claim 100 wherein the antisense oligonucleotide comprises at least one modified sugar moiety or modified linkage.

99. The method of claim 98, wherein the oligonucleotide comprises one or more of:
a. at least one modified nucleotide which comprises a modified sugar moiety;
b. a 2′ modification of a sugar moiety;
c. a 2′-O-methyl, 2′-methoxyethoxy, 2′-O-methoxyethyl, 2′-dimethylaminooxyethoxy, 2′-dimethylaminoethoxyethoxy, 2′-fluoro, or 2′-acetamide modification on every sugar moiety;
d. a locked nucleic acid (LNA) nucleobase; or
e. at least one modified linkage.

100. A method of treating a disease or disorder associated with haploinsufficiency of a gene selected from those listed in Table 1 in a subject in need thereof, the method comprising administering a composition comprising an antisense oligonucleotide sequence complementary to a retained intron of a transcript produced by the gene, wherein the oligonucleotide increases expression of the polypeptide in the cell.

101. The method of claim 100, wherein the disease or disorder is a neurological disease or disorder.
